SEQUENCE LISTING (106 sequences)

SEQ ID NO: 1 - DNA, 627 bp, Solanum tuberosum
gctgacatgtgtgcgagtcaacgggcgagtaaacccgtaaggcgtaaggaagctgattggtgggatccctctgaggggtgcaccgtcgaccgaccttgatcttctgagaagggttctagtgtgagcatacctggaaggctggtttgggactaactggtggaataaacggctctttattattcttgtcaccactcttggcatatttgcaccattggcttccttgaagcgtatagattcattgagatttacgtctgcattatcagtcgccttggctgttgtattactggttgtgactgtgggaattaccatattcaagctaatgaatggaacaattcttatgcccacattgtgtcctgatgtgtatgatataacatcattccttaagctctttacagtggttcctgtactcggtactgcatatatatgccactacaatgtgcactccatagaaaatgaacttgaagacagcacacagatcagagcagtggtgaaaagctctcttgctctttgctcaagtgtgtatgtgttgacgagcctgtttggttttctcctatttggtgatgcaacccttgatgacgtgcttgccaacttcgataccaatcttggaattcccttaagctccttgct

SEQ ID NO: 2 - DNA, 365 bp, Solanum tuberosum
  misc_feature (276)..(276): n is a, c, g, or t
acagccaactcagaactggtacggacaaggggaatccgactgtttaattaaaacaaagcattgcgatggtcccaacggatgtttacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctgtctactatccagcgaaaccacagccaanggaacgggcttggnagaatcagcggggaaagaagaccctgttgagcttgactctagtncgactttgtgaaatgacttgagaggtgtagt

SEQ ID NO: 3 - DNA, 614 bp, Solanum tuberosum
  misc_feature (554)..(554): n is a, c, g, or t
tgggaaaacaccaatggtttatcttaaaaaagtgacagaaggatgtggagcatatatagctgtgaaacaagagatgtttcagcctacttctagcatcaaagacagaccagcattggcaatgatcaatgatgcagaaaagaaaggcttaatatcacctgaaaagacgacgttgattgagccaacatcaggaaatatggggattagtatggcatttatggcagcaatgaaaggctacaaaatggttttgactatgccatcgtacacgagcatggagaggagagtgacaatgagagcatttggagccgatttaatcctcaccgatccaaccaaaggaatgggaggcactgttaagaaggcttatgagcttttggaatctacacctaatgctttcatgcttcaacaattttccaaccctgcaaacactcaggttcattttgacacaactagccctgaaatatgggaagaaactctaggtaatgttgacatatttgtcatgggaataggaagtggaggcactgttactggtgttggacaatatcttaaatccaaaaatncttatgcaaagatatatgaactcgggtcaggactcatcgcaggctgttnggctgctatgt

SEQ ID NO: 4 - DNA, 432 bp, Solanum tuberosum
  misc_feature (409)..(409): n is a, c, g, or t
ttgagcggccgcccgggcaggtacttcgctaaacaagctgagaagaagcagaagaagggagaaggagagttttttgaagaaaagaaagaggagaagaatgtgcttcctcaggagaagaaggatgaccagaaggctgttgatgcagcattgatcaaggccatcgaggctgttcctgaattgaagggttatttgtctgctaggttctcgctcaagtcaggcatgaaaccccatgagcttgtcttttagaccggataattaaactacttttgatgaaattgattttgttagatctattttgttttatgttctgttctgaacctgcgtttagtctttgtattgaagattttgttggttataaacatcttaagttgaaatatgcaatatgagctttgctccttttttgtcccanaaaaaaaaaaaaagaaaaaaaaa

SEQ ID NO: 5 - DNA, 381 bp, Solanum tuberosum
cgggcttggcagaatcagcggggaaagaagaccctgttgagcttgactctagtccgactttgtgaaatgacttgagaggtgtagtataagtgggagccgaaaggcgaaagtgaaataccgctacttttaacgttattttacttattccgtgaatcggaagcggggcactgcccctctttttggacccaaggctcgcttcgcgggccgatccgggcggaagacattgtcaggtggggagtttggctggggcggcacatctgttaaaagataacgcaggtgtcctaagatgagctcaacgagaacagaaatctcgtgtgggacagaagggtaaaagctcgtttgattctgatttccagtacctgcccggggcggccgctcgaa

SEQ ID NO: 6 - DNA, 52 bp, Solanum tuberosum
ggacagtgggaatctcgttaatccattcatgcgcgtcactaattagatgacg

SEQ ID NO: 7 - DNA, 648 bp, Solanum tuberosum
acagaacaaaagatcttgtcatcaagggttctgactggatcgtgaatgaaatgaagaaatctggtcttcgaggacgtggtggtgctggttttccatctggtctcaaatggtccttcatgccaaagacaacagatggccgtccttcatatcttgtcgtcaatgctgatgaaagtgaacctggaacctgtaaagacagggaaatcatgcggcatgacccacacaagttgttggaaggctgtctgattgcagggctggggatgagggctaaagctgcttatatttatatcaggggagagtatgtgaatgaaaggaagagccttcagaaggctagacaagaagcctatgaagctggattgttggggaaaaatgcttgtggatctggttatgattttgacgtatatattcattttggtgctggtgcctatatttgtggtgaagagacggctcttctggagagccttgagggcaaacaaggcaaaccaaggttgaagcctccatttccagctaatgcaggactatatggatgtccaacaactgtcacaaatgttgaaacagtagctgtttcaccaaccatcctaagacgtgggccggagtggtttgctagttttggccggaagaataatgctggggacaagctgttttgcatct

SEQ ID NO: 8 - DNA, 312 bp, Solanum tuberosum
tggaggtgaagatgaaggtgtggatgatcaagctgtcaaggttgtcgatattgttgacacttttagacttcaggagcaacctgctttcgacaagaaacaatttgttacctacattaagagatacatcaagaacctgacacccaagctagaaggagaagcacaagaagcatttaaaaagaacattgaatcagcaactaagttccttttgccaaagctcaaggactttcaattttttgttggtgagagcatgagtgacgacagtgcgctggtgtttgcgttctacaaagatggttcagctgatcctaccttttt

SEQ ID NO: 9 - DNA, 354 bp, Solanum tuberosum
acggtaaatacaatgtttgaatagactgtattgctccatttttaacagaggacttttgtcaaacacttgatctgcaactttttttcaaccagaaatatcaatggctttcacttcaggtttcttctcttcttctttaggaacagttacagtaagcactccattctccatcgccgccttaatttcctccattttcacattctccggcaacctaaacctccttgcaaacttaccactactcctctccatacggtgccattgatcattcttctcctcttgttctttactcctctcaccactaatctgcaaaatccttccttcttcaacttcaactttcacttcctctttcttaatccc

SEQ ID NO: 10 - DNA, 183 bp, Solanum tuberosum
tggaaccaattggaaagccgtctctcgagccatcaatgtccagaatgtagaatcaaacaggagggctctccgtgagcttctcttttgcaccccctggttgtcttcagtatcttagcggtgttatcttgttcgaggaaacactctaccagaagactgctgctggccaagcccttttgttgaagt

SEQ ID NO: 11 - DNA, 376 bp, Solanum tuberosum
gtgaagttttgagcaggctgtttaaagatggtgttgtcaagaggcaagatctattcattacatccaaactctggaacactaatcatgcacctgaagaatgtaccagtcgcactagacaaaactcttaaagacttgcagctggaatacgttgacctgtacctcatccattggcctgtcagtatgaagcctggatcagttgacattaaacctgaaaatcttatgccaaccaacatacccagcatatggaaagcaatggaaaaagtttatgattctgggaaggctagagttattgggtgttagcaatttctctactaagaaactggggagatctttttgcagtagcacgtattcgtcctgctgtcaacccagtggagtg

SEQ ID NO: 12 - DNA, 712 bp, Solanum tuberosum
ggttttatggacatgcgagaaactagagcatctgccatctagagatgccatgcaacgcctcaccaaattacggaacctagaaattaaaggctgcccaaagttagaagaaagttgcacaaatcagagtggcccaaactcccaatggtccaacatttcccatattccaaaaattaaagtaggtggaagcataattcagaacttgaagatgccatgagacatctcaccaaattacagaacttggaaatttaaggctgccgaaagttagaagaaagttgcaccaattgagtagctcaaactccgtgtggtccaacatttcccatattccaaatattgacgtggggatcacgacaattcaggacttgcggtttggttccccttgctggttcaaatgatgagtcatggtgccaaggtctcgatggccttgcctcgcgcactgctgcttactaccaacaaggagctcactttgccaaatgtgagcattcccaacagaccatctgcattggcagtgaaggaagcagcctgaggtttggctcgctatgcagccatttctcaggacagtggtttggtcccaatagtagagccacaaatattgctggatggtgaccatggcattgacaggacttttgaggttgcgcaaaaagtttgggccgaagtttttgtttacctagtagctgaggacaatgtcatgtttgagggtattctcctcaagcga

SEQ ID NO: 13 - DNA, 603 bp, Solanum tuberosum
ggcaacttctctttccctttcgaatatctctcaaagaaaagtcaaagagaaatggcggatcagctcaccgaagatcagatctctgagttcaaggaagccttcagtctttttgacaaggacggagatggttgtatcacaactaaggaacttggaactgtaatgcggtcattggggcagaacccaactgaggctgagcttcaagacatgatcaatgaagttgatgctgatggaaatgggaccattgattttccagagtttcttaacctgatggctcgcaagatgaaggacactgattctgaggaggagctcaaggaagctttcagagtgtttgacaaggatcagaatggattcatctccgcagctgagcttcgtcatgtcatgacaaacctaggcgaaaagcttactgatgaagaggttgatgagatgattcgtgaagctgatgtggatggcgatgggcagatcaactacgacgagttcgtcaaggttatgatggccaagtgagaactgatcaatccaacttaatctttagaagtaaaaatataaaagagaaggaaatggagcagataagtttgtgagatttctattccaaattaggacgtgtta

SEQ ID NO: 14 - DNA, 567 bp, Solanum tuberosum
ggagaccttgacagtagctcaagtggccgccatagctgcacagtcaggtacagacgtgactgtgcagctatcagaggcggcccgagctggtgtcaaggctagtagtgattgggtgatggaagggatgagaaatgggactgatagttatggtgttactactggttttggtgcaacatcacatagaaggaccaaacaaggtgcagccctacagaaagagctaataaggttcttgaatgccggagtctttggcaacgggacggagtcttgccacatgttgccacactccaccacgagggcagctatgctagtgaggatcaacaccctacttcaaggctactcaggcataaggtttgagattttggaagctataacaaagttgttgaacaacaacattactccgtgtttgcccctacgtggcacgatcacagcgtctggtgacttggtaccactgtcctacattgctggtcttttaacaggccggaccaactccaaggccaccgggcctaaaggagaactgttggacgctgtcaacgcgttccaacgggctggtattgagtctggattctt

SEQ ID NO: 15 - DNA, 682 bp, Solanum tuberosum
ggagaccttgacagtagctcaagtggccgccatagctgcacagtcaggtacagacgtgactgtgcagctatcagaggcggcccgagctggtgtcaaggctagtagtgattgggtgatggaagggatgagaaatgggactgatagttatggtgttactactggttttggtgcaacatcacatagaaggaccaaacaaggtgcagccctacagaaagagctaataaggttcttgaatgccggagtctttggcaacgggacggagtcttgccacatgttgccacactccaccacgagggcagctatgctagtgaggatcaacaccctacttcaaggctactcaggcataaggtttgagattttggaagctataacaaagttgttgaacaacaacattactccgtgtttgcccctacgtggcacgatcacagcgtctggtgacttggtaccactgtcctacattgctggtcttttaacaggccggaccaactccaaggccaccgggcctaatggagaactgttggacgctgtcaacgcgttccaacgggctggtattgagtctggattctttgagctgcagcccaaagaaggattagctttagtgaatggtacagctgttggctctggcttggcctcaatggttcttttttgaagctaacattcttgctatactttctgaagtttt

SEQ ID NO: 16 - DNA, 192 bp, Solanum tuberosum
acgagtggctctaatgatgatttcagttcgcatgggagtaaccctaacttcaactcccgagtaaccatcctccgccagctctcttgtcagcacttcgttcaactccgcaaaaaaaactccatctgctacaaactttcgctttttactaatctgaggcgttgccatgttgagattagggtttgatattgattc

SEQ ID NO: 17 - DNA, 430 bp, Solanum tuberosum
gattcccactgtccctgtctactatccagcgaaaccacagccaagggaacgggcttggcagaatcagcggggaaagaagaccctgttgagcttgactctagtccgactttgtgaaatgacttgagaggtgtagtataagtgggagccgaaaggcgaaagtgaaataccactacttttaacgttattttacttattccgtgaatcggaagcggggcactgcccctctttttggacccaaggctcgcttcgcgggccgatccgggcggaagacattgtcaggtggggagtttggctggggcggcacatctgttaaaagataacgcaggtgtcctaagatgagctcgacgagaacagaaatctcgtgtggaacagaagggtaaaagctcgtttgattctgatttccagtacctgcccggggcggccgctcgaa

SEQ ID NO: 18 - DNA, 633 bp, Solanum tuberosum
  misc_feature (512)..(512): n is a, c, g, or t
gttatttctcgcagcacttttcctctctctctcctacctctccggcgcggcgggtaacgccgcagcctctaccagtttcataaaaagctcttgtaaatccactacttttcccgatgtatgcgttgcttccctttccggttacgcacaagccattaaaaacagccaattgcagctggtgaaaacggctctgtccgtgagcctagataaagccgaatcgacaaaaggattcgttagcaagctgttgaaattcaagggattgaaaccgagagagtatgcagctatcaaggattgcgtggaggaaacgaatgatagtgtagatcggcttagcagatctgtgaatgagctgaaaggtttggatcgtaaccatgggaaagccgatttccagtggcatatgagtaacgtagagacatgggtaagtgctgctattacagatgagaatacttgcactgatggatttgccggccgagctttgaatggtaaaattaaggcttccattagaagtcacatagctnatctagctcaggttactagcaatgcactggccttaatcaaccaatatgctgcaaatcattgattaattggctacgttaattatgtttagagtgttttgagcttaattatggttttttgcct

SEQ ID NO: 19 - DNA, 198 bp, Solanum tuberosum
accagccccaaaagctacttatggtatgagttccacacacaaatatcacaaaataagatcaaaaccacataatgtccacaagttgacaagcctttcaaatagagcttaacaacagaccaatatcacgtcttagccgacctcctgtcccaacaaatattaacatttctcttcgacaaagtaaagagaacatccaatgtt

SEQ ID NO: 20 - DNA, 84 bp, Solanum tuberosum
acgacccatgactttaatcacaattgcatgcttggttacagattccatccttccggtgagaaaaatctgctgctgtatgttgct

SEQ ID NO: 21 - DNA, 330 bp, Solanum tuberosum
gtcctgaccgaactgggcagtatctgtacagaaaggatatatccatatgtaaacacttatgacggaaaaacaatccattaccctgatcccatcatcaagtacagcttgacgataaatcctgaccgagcggggggtataatcctctgcagacaatgagtcctgaccgaccgaaccaggttagatctggaagatggtaatgggtctcatgaccaaacactccttaatgaacgtgctaccctccataggactactggacagatggatggtgtgatttctcaagctgaagaaacaataaagacacttatgtttcaaagatcaacatttggtggc

SEQ ID NO: 22 - DNA, 617 bp, Solanum tuberosum
  misc_feature (440)..(440): n is a, c, g, or t
aagacgctttgtttgctcggagtttcttcctatcagagagcaattttgcaaacctctttagagcttcctcataggtttcaggactgtctttggaaactggtttcttcaataatgtttcaccaacttgtactaataattccatattagcctcagacgcatcatccatttcagtagttgtgccatttaatgcattttcttgaaccctgaggtaattgttttgtgaatgacgagcttgaaaaacagtagaaatgtaataatcagtcatgtaagaacttgctgcattagtcatttgctgtatagctaacatccatcgtagaggaccccatttagctgcctcttctgctgtatatgttttatcaaactctgaattagtgccagtgcctaatgagagcaacaacatttgcttgtaatccaatgacttaattgaagcaaatgctggnatcctcttggtgcaagtcttcggttgnaacgcttaagggattaataacgccggatcaaccaacaagtagcaacaggacccatcaacaaggattgactcnatattgtagcaccaatactaggtntgaagttacaaangtgatgtnggggggaaatntttttggagcttgctgcttgtg

SEQ ID NO: 23 - DNA, 321 bp, Solanum tuberosum
  misc_feature (172)..(172): n is a, c, g, or t
tcagcccaccgacagctgacgatgagtcctgaccgatcggtgggtagaatcctttgcagacgaatcggagcgacaaagggctgaatctcagtggatcgtgacgatgagtcctgaccgaaggatcttctgccctgcccctgaaatttattctgggggttgctctagtttcagngatcctgggaataattcttggtaaaagatattagtntcaaggtttcccagttttggtcttgtttttagaatggagttcttttctggntactttcttctntacccctttgtgattttcggaaaagnacttggtcantattgcntggcata

SEQ ID NO: 24 - DNA, 118 bp, Solanum tuberosum
ggagccatcacttgcaattttgaaattccagacatattaactaaagtggtcgtcaagagaacaacgaagaagatagcaaacttcatctgtgaatttttggcggccattatggttttgg

SEQ ID NO: 25 - DNA, 269 bp, Solanum tuberosum
  misc_feature (5)..(5): n is a, c, g, or t
gtagntctgnttnaccggcgtngttacaattgcttcagntggggctgggataattgttttagcgggcgacaagngttgntgtnctgaagtnatagntagaaagaggggttggtaatctaattcctcttagcggtctcatagtagagtgagtgatcctcggntaaccatctatgcagttgatgnagttcagantcaacangggatcgcngancgaagggagtgtatnnctgntttttttttccactcccctgtccgcatcgagggagagg

SEQ ID NO: 26 - DNA, 100 bp, Solanum tuberosum
  misc_feature (90)..(90): n is a, c, g, or t
agagccatcacttgtatattaactaaagtggtcgtcaagagaacaacgaagaagatagcaaacttcatctctgaatttttggcagccatnatggtttngg

SEQ ID NO: 27 - DNA, 113 bp, Solanum tuberosum
tgacgataaggactttattccatttgtgttcatcaaggcgtagaatgctaattagctggctagcttgtagctttctaaataaagcgcatatatccttttattgctccatggaa

SEQ ID NO: 28 - DNA, 606 bp, Solanum tuberosum
  misc_feature (478)..(478): n is a, c, g, or t
tatttccttagcaagctttgttggtacaccaataagttctggccagaattgttttccattgcattcaaattcctttagaagttctatgacttctggtccatcactttcttttcgtgccaagagagtttcaaaggattctacccaccgctcggtcaggactccatcgtcaatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagangcggtttgcgtattgggcgctcttccgcttcctcgctcactgactcgctgcgctcggtcgttcggctgcggcgaacggtatcagctcactcaaaggcggtaatacggttatccacagaaatcaggggg

SEQ ID NO: 29 - DNA, 142 bp, Solanum tuberosum
gcaatggaaaactaagctggccagaacttattggtgtaccaacaaagcttgctaaggggataattgagaaggagaattcactcataagtaatgttcatatattactgaatggttctccagtcacattggattttcgttgtgt

SEQ ID NO: 30 - DNA, 100 bp, Solanum tuberosum
atagccatcacttgcatattaactaaagcggtcgtcaagagaacaacgaagaagatagcaaacttcatctctgaatttttggcagccattatggttttgg

SEQ ID NO: 31 - DNA, 450 bp, Solanum tuberosum
ccagcgaaaccacagccaagggaacgggcttggcagaatcagcggggaaagaagaccctgttgagcttgactctagtccgactttgtgaaatgacttgagaggtgtagtataagtgggagccgaaaggcgaaagtgaaataccactacttttaacgttattttacttattccgtgaatcggaagcggggcactgcccccctttttggacccaaggctcgcttcgcgggccgatccgggcggaagacattgtcaggtggggagtttggctggggcggcacatctgttaaaagataacgcaggtgtcctaagatgagctcaacgagaacagaaatctcgtgtggaacagaagggtaaaagctcgtttgattctgatttccagtacgaatacgaaccgtgaaagcgtggcctaacgatcctttagaccttcggaattcggtcaggactcatca

SEQ ID NO: 32 - DNA, 271 bp, Solanum tuberosum
ccgctaaggagtgtgtaacaactcacctgccgaatcaactagccccgaaaatggatggcgcttaagcgcgcgacctacacccggccgtcggggcaagtgccaagccccgatgagtaggagggcgcggcggtcgctgcaaaaccttgggcgtgagcctgggcggagcggccgtcggtgcagatcttggtggtagtagcaaatattcaaatgagaactttgaaggccgaagaggggaaaggttccatgtgaacggcacttgcacatgggttag

SEQ ID NO: 33 - DNA, 113 bp, Solanum tuberosum
ttacatggagcgatagaaggatatatccactctatttagaaagctgcaagctagccagctaattagcattctacgccttgatgaacacaaatggaataaagtccttatcgtcg

SEQ ID NO: 34 - DNA, 454 bp, Solanum tuberosum
gtatctacaattagaatggcattcctaaaggcatttgcagctttgctcattgcatcacttgtgcttgtccatttcacttatgcccttcaagaggtaattaacagcaaaccaccagcaccaagtcctcaacctctaaagccaatagattgtacgggatcttgtaaaatgaggtgcagcaaatcgtctcggcaaaagctatgcaacagagcatgtggaacctgctgccagagatgtaagtgcgtgccaccagggacttctggaaattacgaagcatgcccttgctatttcaaccttactactcataacagtacccgcaaatgtccttaaaatccgtagcataattttgttgttctgcaaattcaaatgtacctcttattaaatactactattattatgttgtgtactatgtcagctcttgattttgaaatttaagggtaaaattgaaggttgagag

SEQ ID NO: 35 - DNA, 789 bp, Solanum tuberosum
gattttattacaatgttgaaaatcaagattgattatttttttctgactttcattttagtattttcttcaccactagtggcttcctaaaaatggaatatgccttgtctaaaagttagcttttttctctgattacttcattaaccatgttcttcaatttttgcttaacatttcttctctgagtatcaggatagttggatgattgaaatgtgttatggatgtgaatgttaaagcttggagctggtgaactttttgttacgatgccgggtgtatgctggaaaatctatgatgtgctaaaccggtagataatatttcacaatgaggccttctcaaaaccctgaggatatcaacgagttcaacagattctataaccaacccatagagtatcaagaatcctatttcctgccttctattaacaatccaaacaacaaccaatcgtactatgctgatgtctctgccttaaaacccgaccaacactgctatgttgaatcatccacgggaaattctagtgaacttgtttccgattcacctcctattgttaatttcaaccccatgatgcagcaaggttctgattcatggcgttcagacatgcatcattctcctgatgatacatttcaatcatcgggtaacagttcgtgctacactagtgatgttactgacctgaagcacaagttaagggaacttgaaactgcaatgctggggccagattcagaaagcatggaatcatataatactattccctgtggctgcagccaatcaagtttcatcggaagtcggataaattggttggaa

SEQ ID NO: 36 - DNA, 706 bp, Solanum tuberosum
gttatgtttgtgtttgcttcccattgtggtgttttcatcaactttcacttcccaaaatctcattgacctacccagtgaatctcctctacctaagccggtacttgacacaaatggtaaagaactcaatcctgattcgagttatcgcattatttccattggtaggggtgccttaggtggtgatgtatacctaggaaagtccccaaattcagatgccccttgtccagatggcgtattccgttacaattccgatgttggacctagcggtacacccgttagattcattcctttatctggaggtatatttgaagatcaactactcaacatacaattcaatattccaacagtgaaattgtgtgttagttatacaatttggaaagtcggaaatctaaatgcatattttaggacgatgttgttggagacgggaggaaccatagggcaagcagatagcagctatttcaagattgttaaattatcaaattttggttacaacttattgtattgccctattactcccccttttctttgtccattttgtcgtgatgataacttctgtgcaaaggtgggtgtagttattcaaaatggaaaaaggcgtttggctcttgtcaacgaaaatcctcttgatgtcttattccacggaagtttagtaacaaataatgcctgcagatagactatactatgttttagcctgcctgctggctagctacta

SEQ ID NO: 37 - DNA, 562 bp, Solanum tuberosum
gatctaatggagtcaaaaggtggtaaaaagaagtctagtagtagtagttccttattttacgaagctcccctcggctacagcattgaagacgttcgaccaaacggtggagtgaagaagttcagatcagctgcttactccaactgcgcgcgcaaaccatcctgatatttcctaaacttccttccttaaagcgtcaatagacgcaaccaaaaaaaaaacaaaaaaaaaaaaattcttctttcagtttcttcttttgtcaagccctcactccttttcttccttcttttactacttcctgcttttgcactcattgctttgaacattttccgctttaacttcctttgctgctgtgaactttttcataatggaaatggacttgccagtttctgccattggttttgaaggttttgaaaagaggctcgaaatttcttttgtcgagcctggtctgtttgctgatcctaatggaaaaggacttcgatctctctcaaagcacagttggatgaaattctcggacctgctgagtgcaccattggtggatacctatcaaatgactatgttgattcct

SEQ ID NO: 38 - DNA, 681 bp, Solanum tuberosum
gctttaattctctcctgtcaatggcggatttggtttcacatattcataaaaccttcctatcattttccctttatttcttcttctctaatttttagcctttttttctcatttttttaaccccttttttggcctataatttcaaggaaaaaatcaacaattttcaggagttgatcttataaagtatcatgggtaattgctgtgtgaaaccgggtaaatctgctgaaaaaaagaacaaaaagaacaacagcaaacccaaccctttttcaattgattatgggggtactaaacatgcctctgggagtggaaataagctagttgtattaaaagagccaacagggcaaaatatacatgataagtatgatttgggtcgtgagctcggaagaggagaatttggggttacttatctatgtactgacttggaaggaggagagaaatatgcctgcaaatcgatatctaagaagaagctaaggactgcagtggatattgatgatgttaggagagaggttgagatcatgaaacatttgcctgtgcatccgaatattgtgaccttgaaagatacttatgaggatgataatgcagtgcacattgttatggaattgtgtgagggtggggagttgtttgatcgaatcgttgctagaggccactatacagagaaggcagctgctggaattttgaagactg

SEQ ID NO: 39 - DNA, 658 bp, Solanum tuberosum
gagaaaacatggattttctcattaataaaaaggatttaagagttacaattggtagaaaaagcaacagaaacctaataacaacacgtttcaactgccaaaaaaaacgaacagttctttttaaacttatatacaaaaatcagccattagttctgtcactctcttaatctctctttcacaaaattaggttatcgtcgtcatcaaattaaacttccatgggtggcgcaaggttaagatcaagatcaagtccttttttcccgtaacgattctcttccacaacggaagacgaatccgacacactagaaacaactccggcaccgacaccggcgaactccatcgtcgccggcacaacctggtacggcctgctaacaactcccggtctccacatagaatcaaacagcaaaaccggctgaccgttcggcagtaccgccaccgtcggctgctgatggaaaaccgggtagccgacacctccacgtccaccttcagcaacggcgccaagacggcgcgtgagatccaactctagcggcgcgtgtggcgtatgaggcgcgtgaacaacagtttctccgctagatgactcgacggtgctgcttccacaagggctctgattattactattaatattaatctccgccggaaaggggaaattcgtcttagctttagg

SEQ ID NO: 40 - DNA, 744 bp, Solanum tuberosum
gcgaaaggcgaaagtgaaataccactacttttaacgttattttacttattccgtgaatcggaagcggggcactgcccctctttttggacccaaggctcgcttcgcgggccgatccgggcggaagacattgtcaggtggggagtttggctggggcggcacatctgttaaaagataacgcaggtgtcctaagatgagctcaacgagaacagaaatctcgtgtggaacagaagggtaaaagctcgtttgattctgatttccagtacgaatacgaaccgtgaaagcgtggcctaacgatcctttagaccttcggaattcgaagctagaggtgtcagaaaagtcatcccagagaagtaccttgacgagaagacaatcttccaccttaacccatctggccgattcgttattggtggacctcatggtgatgctggtctcactggtcgtaaaatcatcatcgacacttatggtggttggggtgctcatggtggtggtgctttctctggcaaggacccaaccaaggttgacaggagtggtgcatacatcgtaaggcaggctgcaaagagtattgtagctagtggactcgctcgcagatgcatcgtgcaggtttcttatgccatcggtgtgcctgagccattgtccgtattcgttgacacctatggcactgggaaagatccccgacagggacattttgaaagatcgttaaggagaacttcgacttcagacctggaatgatgtccattaatttgg

SEQ ID NO: 41 - DNA, 559 bp, Solanum tuberosum
agggtaaattgcgaggattcatatagctgaccagactagtttaaggcataataatagtaattgtatgttatgccattattcactcagaaacattatagaactaatacctcacttgctaagctacttcatgaaaaaaaataattaaaacacatcacttgctatgttctggctttgcagaaccgtaataggtaagtgagaatgtgataagaagctcaatttttctgaagaatcttgttcttcatgtcttgtatgaattggttgagattcttctctgaagatccctcatcaaaaagtgcattttgaagtatcatctttgttttatcgatattctccctcgattcatctgagttattcattatctttccaatattctccgctactacctccctcctgatcaatcctctagtgcaaagattcacaccgatcctccaatcatcaaccactaatttcctgttagtaagttgatcccccgaaacaggaaagcacaccattggaactttacaccatatactctccaagattgaattccacccacagtgagtaagaaaccccccaactg

SEQ ID NO: 42 - DNA, 575 bp, Solanum tuberosum
aaatctggttacttctagtccttaataatcatatatagtggtggtgggtgaaatgtattaataaaaactatacaaagaaagaatcttttttgtagatttgaagtgggttttttgtgggtggtgttaaagattcaagaaaatggggaatgttaatggaagagaagaaaatgagggcaatatcccatctggggttgaaggggttgattctggtggggtccaagatatcatgaccgttcatcaggttgatggagagttcatgggtcaatctccaccgtccagtcctagggcatctcgttcacctttgatgtttcgacctgagatgccagtggttcctctacagagacctgatgaggggcacggcccaagcatttcatggtcgcaaaccacctcaggttatgaagaaccttgtgacgagcaaggagttccaacacttatatcttggactcttgatggcaaagaagttgctgtggagggatcatgggataattggaagtcaaggatgcctctgcaaaaatcagggaaggatttcaccattttgaaggtgcttccttcaagggtataccaatatatgttta

SEQ ID NO: 43 - DNA, 710 bp, Solanum tuberosum
ggccagtagtcatatgcttgtctcaaagattaagccatgcatgtgtaagtatgaacaaattcagactgtgaaactgcgaatggctcattaaatcagttatagtttgtttgatggtatctactactcggataaccgtagtaattctagagctaatacgtgcaacaaaccccgacttctggaagggatgcatttattagataaaaggtcgacgcgggctctgcccgttgctgcgatgattcatgataactcgacggatcgcacggccatcgtgccggcgacgcatcattcaaatttctgccctatcaactttcgatggtaggatagtggcctaccatggtggtgacgggtgacggagaattagggttcgattccggagagggagcctgagaaacggctaccacatccaaggaaggcagcaggcgcgcaaattacccaatcctgacacggggaggtagtgacaataaataacaataccgggctctatgagtctggtaattggaatgaggaccgataacaccatcaggtacaacgccaagcgcaggcactggaggcgtaccaagttaggattttgaggtggatggcttggagttctcttcaagttccattgaaaaatttatgggattttggttacttaccgttttgtttagtaggagcttgtttctgaaacgaaatctagtagagttgttttctactgatgttttgggaaaata

SEQ ID NO: 44 - DNA, 681 bp, Solanum tuberosum
gcctgttcgttcttccttccagaataattgtaatgacttctgcaagtgcaaatgagaattgccttggttgggcagcgagagatccgtctggagttctttcaccttatgaattcaatcggagggcagtcagcagtgatgatgttctgctagacattgcattttgtgggatgtgtttcgctgatgttatctggaccaggaatataattggaacttcaaaatatcctcttgtgccaggacatgaaattacgggaattgtgagagaggttggtacggatgttaagcacttcaaagttggcgaccatgttggagttggaacatatgttaattcatgcagggaatgtgaatattgttgtgatgaattagaggttcattgctcagaaggagcaataaacacttttgatggtatagacgtcgatggtacagttactaaagggggatactccagttacattgtcgtcaatgaaaggtactgctttagaatacctgaaaattacccgcttgcatatgcagcacctttgctatgtgctggaattactgtttatactccgatgataagacataacatgaaccaaccggggaaatctttaggtgtcgttggattaggtggtctaggccacttagcagtgaagttgggaaggcttttggattaaaagtaacggtattcagtacaagtatatctaa

SEQ ID NO: 45 - DNA, 703 bp, Solanum tuberosum
ggcttcttcatcatggaactttctaccattaattcacctaaaactcatcacaataaactaatttcttctgatgatccttcaccgccaacgacgacactgctacttcaaagatccaagggtggtcttgtaaccatgccatttattatagcaaatgaggcattggagagggtggcaacttttggggttttaccaaatatgacattttatttgatgaaagagtatagtatgggagtgacaaatgcacaaaatttactcttctattggtcgtcagctaccaatttcttgccacttattggagctttcatagcagattcttatcttggacgcttccttaccattggatttggttccatcttcagtttcttggggacaattgtgttgtggttaactgcaatgattccgaaagcaaggcctcccccttgcaatcttctagtgtcaggtcaggcatgtaaatccgcaacaacagctcaatatacaatcttgatctcctcgtttatgcttatgtcgattggcgctggaggtattagaccttgttcgttagcatttggtgcaaatcagtttgataagcgcgatactaatggtaatatcaaacgcgtgttggagacttattctaactggtactatgctgccgtcatgatctctgttcttattgctatgactggcattgcatatcttcaagaccatatgggatggaaaatcggat

SEQ ID NO: 46 - DNA, 648 bp, Solanum tuberosum
  misc_feature (546)..(546): n is a, c, g, or t
tggacaaacagaggatatatcaagggaagctggaaaaatggctttatatatgattcctcaattatttgcatatgctatgaattttccaatagccaaattcttacagtctcaaagcaagattatggcgatggcatggattgcagcaatagctttggtttttcatacattttttagctggttgcttatgttgaagttggggtggggccttgtcggtgccgcggtggtgctcaactcatcatggtggttcatagtggtggcgcaaatgttatatatatttagtggtacttgtggacaagcatggtcaggtttctcatggaaggcttttcttaatctatggggctttgttaggctatctcttgcctctgctgcatgctatgcttggagacttggtactttatggcgttagtcctgttcgctggttatttgaagaatgcagaagtcgctgttgatgccttgtcattagcatgaaacatactacgatgggcagttatggcagctattggatgccaatgcagctataagtgttaggggggcaaaatgaactcngggcanctnaatccaaaaaccgggtaaattttcaggggggggggggtggggttctcttcattcttgaatgggcttctcttatcggngntttctactagccgg

SEQ ID NO: 47 - DNA, 78 bp, Solanum tuberosum
agcatcttccttattgttccgttggcttttgtagatcaaagggttacatgatttgtatggtttttagtcacatgtgat

SEQ ID NO: 48 - DNA, 131 bp, Solanum tuberosum
aggtcggatggctattatgcaactgacttgagcaaggcgttcaacactacttcttttgttggcaaagctggaattgctcctctctccatcacagtcaatgcaccacaagcattagcaaaactaagggcatc

SEQ ID NO: 49 - DNA, 192 bp, Solanum tuberosum
acttggactagggtatatctgtggggtttgattgagcaagactgtggatccttttcttcatctcattgggatttgcaccaactatcttgtcaactggcactccacctttcagcagcagaaatgttggcatggctttcacctcatattttctggccacctccttgacctcatccacatcaactgtgagaaaag

SEQ ID NO: 50 - DNA, 306 bp, Solanum tuberosum
acacagtttctcggcctgcttgtaatctttcaatttcaacttgcaggctgcattatttaagttgcaggtgatcttcaatgctttagactgtttcttttcctcctcgctgaaggaagtatcatactcgatgaatttagcagccttctcatatctcttagatgcccttgcgtatttaccagccttgaacagtgcattcccttcttctttctttttgccagcagactcaattttctccggggtattcatatcccatgattccttgtccttgacaaaagaaacaagctcaacctcataatgcacagttga

SEQ ID NO: 51 - DNA, 246 bp, Solanum tuberosum
aaggagctgctgccaatcaaaatgcaaaagattcttcctccgtcacccagaggcaccttggaattacctgtaatagtaacagttcatcagttgtagcatcaaaaaagaaaaataggaaaggtggactctccatgtttctgaacggtgctcttgatgaagttcctaaagttgtagttcccccaccagttgtgcaaaaaagtgaagtccctgcttggggtggtgctaaggttgcgaaaggctctgcat

SEQ ID NO: 52 - DNA, 587 bp, Solanum tuberosum
actggagaaaacccatgcattacaagtcatacactgtagcatttcctagcatgaactcttaagcctgtctgttcctcttcttggcacctgacttggtgaccttgagactcctgttcacaacactcaaccttgcaagggctgccttcttcagatctggcctgtagtagttgtctccaacctggtttgttacagccttggccattctgcggaactccttcttcatcacagatttgttcagcaaacttgaaggcatgttttgcttcttggttttcgatgtagcaagcaacacagatttatcttttccctcaggctggatagtcactgtcttcttgtttgctaggccagagtgcttgtaggagtgaaggttgcagagattgttaggttctttgctgaagacgacgccagcgctaccattaccgaactccttcaccaaaaaggagttgttcttcttcacgatctcccatactagttgccctggaactgttgtcattgcaaatgattcctagggtttgctcagacggcgggaagaggagacctcggccgcgaccaagctactcttcgagaaatacaggatgagacatattatg

SEQ ID NO: 53 - DNA, 287 bp, Solanum tuberosum
actgttttgatttccatatgcttcgggatctagcttgctttttggaggaaattcttgaagtctactaattataacaggattaactccagctagcatttctcttgcgaattcttcatcagtcctccacgcggttttactatctttaatcactagaggagttggaaatctcaatattccttcaccgtctgttcgaaggagttctcttatcatctccagaggtatagcagcagtgagagccttaaataaaggtccttgaggaagtttgatccctccttcatatagtctaa

SEQ ID NO: 54 - DNA, 477 bp, Solanum tuberosum
actccagatgtggccgcattaacgactgcatcatagtgagatgtatcacctctgatcacagccccaatgcagagtattgcttgatatttctgtgactttccaagaagctgtgcagtcacgccgatttcaaaacaaccaggaacccacacaacatcaatatcttcctctctaaccgagtaattcttgaaagtctccaaagctccctccaaaagcttcttggtgatcagatcattaaaacgtgcaaccacaatagcaaaccgatgccctttagcagagataacagaaccattcaactgacgaacagcatctgttttaaacaaatccgacccgtttcgatccacacaatgtctctcaattgcagacccgaaacctatagattgagtgaatgacagtgcaggaggtgaagaatttacactttgtctatggaaagacaaactgtatagctgacgatgagattgctgaggattcacagatgct

SEQ ID NO: 55 - DNA, 308 bp, Solanum tuberosum
acaacaaaaagaagttaaaccttataaataagtctggccaaccagttctagcaagcagaatacagatgaaaggatcattcacttgggaatgtcctgatacgcagcatcgaccaaagccagattacaactgtggcctcctcctctgctccttaatcatcagcatcagaacgtggcaaggggctgcgggaaccagcacgtctaccccgcggtgatatactctttggagttggagaccggtccttgtgattacgtccatctggactagcatccctaggaggagttgttcttcgtggagaagggctccttcg

SEQ ID NO: 56 - DNA, 293 bp, Solanum tuberosum
tctcaagcaaaaatggctccaaagaagggtgttgcagtagcagcaaagaagaagccagagaaggcgaaggtggtgaatccactgttcgagaagcggccaaagcagttcggtatcggtggagcgctgccgccgaagaaggatgtaacaaggaatgtaagatggcctcggaatgttaccctacaaaggaagaagagaattctcaagatgcgattgaaggttcctcctgctctaaaccagttcaccaaaacccttgataagaatctcgctacaaaccttttcaagatgcttcttaa

SEQ ID NO: 57 - DNA, 465 bp, Solanum tuberosum
accatccttgctaagatccagaaaagaacaccaatgctgtgctttcctttgttattggcagggataccaatgtcgacatagcgcattggagaatcagtgtcacagaaagcaatagttgggatgttcccaagtgcagcttccttgattggctgatggtcagttcttggatcagtgagaatcaagagtcgcggctcactgtatgaggtctgaagctgattggtaaatgttccaggggtatgacgtccagcaatagcatgtgcaccagtatattgggcgaatttcaagacagctctctgtccatatggcctggcagactgaacaatgatgtcctgaggattctcaatagcaacaatcaccctggcagccatttgaagcttttcccatgtctttccaaggttgatgatgtagataccatcgttacggcgcttgaaagcgtatcgctccatttggaagtcacaattctta

SEQ ID NO: 58 - DNA, 611 bp, Solanum tuberosum
accattgaagctttctacaatgccaatttgggcattacaaaaaagccggtgccagattttagcttttatgaccgatcagccccaatctacacccaacctcgatatctaccaccatcaaaaatgcttgatgctgatgtcacagatagtgtcattggtgaaggttgtgtgatcaagaactgtaagattcaccattccgtggttgggctcagatcatgcatatcagagggagcaattatagaagactcacttttgatgggggcagattactatgagactgatgctgacaggaagctgctggctgcaaagggcagtgtcccaattggcatcggcaagaattgtcacattaaaagaaccattatcgacaagaatgcccgtataggggacaatgtgaagatcattaacaaagacaacgttcaagaagcggctagggaaacagatggatacttcatcaagagtgggattgtcaccgtcatcaaggatgctttgattccaagtggaatcgtcatctgaaggaatgcgttttaacttggttgtcctcctagattttggctaaacagccatgaggtagaaacgtgctgaacttttattttcctgtgctgtagaaatctagt

SEQ ID NO: 59 - DNA, 118 bp, Solanum tuberosum
gtttggctggggcggcacatctgttaaaagataacgcaggtgtcctaagatgagctcaacgagaacagaaatctcgtgtggaacagaagggtaaaagctcgtttgattctgatttcca

SEQ ID NO: 60 - DNA, 314 bp, Solanum tuberosum
acatcctcagaaattatctatgccagaatgccattgatgcagcagaacaaggcgattttggggaggttcggcagttgttgaaggttatgcaatgtccatttgatgaacaacctggcatggagaaatatgcacgcttgcctccagcttgggctcatcgtccgggtgtatgcatgctttcttgttcctcataagttttcagggcgtatgcttatgctttcttgttcctcataagatttccactatgtaatacaagagtttcacttgtatatatataatagaaatcagttcttcgttcttgcagtaatgtgtaaaaa

SEQ ID NO: 61 - DNA, 245 bp, Solanum tuberosum
acttgacttgctgctactttcatcttcatttgtgctttggcttcttttcttttcctcttcaactccttctcctcagcctttagctgttgaagctgtcccagcaatagatccgccgcctccgaaattgtcttcccttgaacttgatcaaccaaatcagcatcacaaccaaaccctaatcccatctcattcaaattcgacaaattcctcgtcaaacccttgagtaatttcatcttcttcttcgtttt

SEQ ID NO: 62 - DNA, 202 bp, Solanum tuberosum
actgaactcccttacacatatccaccaccatattgggaccagttgtctcaggaccaaagcaccaaatcttctttgcaagatctttgtcccaaccaaactcctcagccaagatcttggaacgaaccttggggtcatccctagggccaatgcgtccgtcatcaatagcctcagcaagcccttcctccattggtctagcctccat

SEQ ID NO: 63 - DNA, 406 bp, Solanum tuberosum
ttccgctgctagttctgcttgtgatcacattcgtgactgggtccttggaactccagagggcacttttgtttctatgggtgtttactctgatgggtcatacaatgtcccagctgggcttatttattccttcccagttacatgcaaaaatggagaatggtccattgttcaaggtcttccaattgacgagttctcaagaaagaagttggacttgacagccgaggaactatctgaggaaaaagcattggcttactcctgccttacttaaattacccaaatatcctgaattgccttttcgacatatgaggagtgtatgtggtaatagaagtggcttcagttttgaataaggccattgttgattcttgagggaagtgatgatgatgggaatactgctttgtcagttgtttat

SEQ ID NO: 64 - DNA, 373 bp, Solanum tuberosum
accaatcattccagcatgagcacacaagtagaagtcataattccttgggtgacagactttgttgtctatgattgtgcctggaggaacattgttaggatcattaggctggaaaaacttggtatgatgattcttctgggcaactatcaccacaaacttgggtgaccacttctcatcaagaaatttacaagcctcaataatctgatccagctcaacattcagaacttgactaaactgagattcactgacaccatccctgaatattattatatgctcgggcttcctttttccagagctcacataaaaatctagcaaagcctccctcattattccctcatcttcagtatcagaagcacgtttaaacaagttgtctatc

SEQ ID NO: 65 - DNA, 287 bp, Solanum tuberosum
tgaagagaagcgatattggcagcgatacatttacttatggattaattatgcattatatgaagagcttgatgcagaagacatggaaaggaccagagatgtctacagggagtgtcttaagctgattccgcatcagaagttttcatttgcaaagatttggttgttggctgcccagtttgagcttcggcagttaagactcaaggaggcgcgacttttacttggagaagcaattggaagggctcctaaagacaagatatttaagaagtacctgcccggggcggccgctcgaa

SEQ ID NO: 66 - DNA, 383 bp, Solanum tuberosum
acatctccttcttcttctccctctccgtcttcaaagacgcctggtgcttggtcaaacgtttgcggatggccctagttttctttgggcgcaggtcaagaggcaggtatttcttgttcttgtaaacttccctaagagcagccttttgcttctgtgaaattacagtcagaacctgggcaatagacaaccttaccaccttgattttggagagcttgttaggagcaccaccagtgaccttcgcaacacgaagaagagcaagctctgccttcaaatccttcaactgagtcagtagatcaggcttcgacttgttcctcagctcatgaaccttgattctggccattgttggtgttggtggagatagctacagaggcggcagcggcggctcg

SEQ ID NO: 67 - DNA, 191 bp, Solanum tuberosum
cccatagttcctcagtagccgtcccttgcaggttgaaggaggaggaagatgtctcacaggaagtttgagcacccaagacacggttctttgggatttctccccaggaagcgtgctgccagacacaggggaaaggtgaaggcattcccaaaggatgacccaagcaagccttgcaagctaactgccttcttggg

SEQ ID NO: 68 - DNA, 337 bp, Solanum tuberosum
accacaggaggatctctgagggatttggtctgctatggcatccaagcggattctcaaggagctgaaggatctccagaaagatcctcctacctcttgcagtgctggcccagtagctgaagatatgtttcattggcgagcaacaatcatgggtccagctgacagtccctattctggtggagtgtttcttgttactattcattttccacctgactatccgttcaagcctccaaaggtagctttcaggacaaaggttttccacccgaacatcaatagcaatggcagcatatgcctagacattttgaaggaacagtggagccctgcacttaccatctccaag

SEQ ID NO: 69 - DNA, 419 bp, Solanum tuberosum
atgaatggattaacgagattcccactgtccctgtctactatccagcgaaaccacagccaagggaacgggcttggcagaatcagcggggaaagaagaccctgttgagcttgactctagtccgactttgtgaaatgacttgagaggtgtagtataagtgggagccgaaaggcgaaagtgaaataccactacttttaacgttattttacttattccgtgaatcggaagcggggcactgccccctttttggacccaaggctcgcttcgcgggccgatccgggcggaagacattgtcaggtggggagtttggctggggcggcacatctgttaaaagataacgcaggtgtcctaagatgagctcaacgagaacagaaatctcgtgtggaacagaagggtaaaagctcgtttgattctgatttcca

SEQ ID NO: 70 - DNA, 613 bp, Solanum tuberosum
accagtccagaggaatctacttgaatgccaaggttgctttctgcatccataacattgcctaccaaggtcgattttctttctctgacctccctcttctcaatcttcctgatgaattcaggggttcttttgatttcattgatggatatgagaagcctgttaagggtaggaaaatcaactggatgaaggctgggatattagaatcacatagggtggttacagtgagcccatactatgcccaagaacttgtctctgctgttgacaagggagttgaattggacagtgtccttcgtaagacttgcataactgggattgtgaatggcatggatacacaagagtggaacccagcgactgacaaatacacagatgtcaaatacgatataaccactgtcatggacgcaaaacctttactaaaggaggctcttcaagcagcagttggcttgcctgttgacaagaagatccctttgattggcttcatcggcagacttgaggagcagaaaggttcagatattcttgttgctgcaattcacaagttcatcggattggatgttcaaattgtagtccttggaactggcaaaaaggagtttgagcaggagattgaacagctcgaagtgtt

SEQ ID NO: 71 - DNA, 358 bp, Solanum tuberosum
tgataagcccgtgaagcaggaagaaccacagaagaaaactaaggttgtcacttttgaaacgcccaatgacaaagcgaaagattctgataagtccaaaggacaagaaaagcccactgtaattgataagcccaaaggacaagaaaagcccactgtaattgataagcctaaggacaaaccaaaagatcccgaaaagcccaaggacggtcctacatctgcagcaatggtggcaaccccatggatatccgaaccaatgatgatgatgccaccagttcacggatacccgcaggcaatgcctggttgtggctgtgggcaatgccattataccggtggcccgtgttaccagtattacggaacacct

SEQ ID NO: 72 - DNA, 291 bp, Solanum tuberosum
actatcttggttgaaagactgacccctctgaagaaacagttgcaggaaaaaaatggttctgttgattggccaacgctgattcgccaggcacaaaaacaatcaataaacttagcagcaaattcttattatgtgccagaattctcgaggtatttgaccttcggcgctgctgtcagtgaggtggagatagatgttctgactggagagactaccattttgcagtcggatattatttacgactgcggacagagcttgaatgcagctgttgatttgggacagattgaaggtgctttt

SEQ ID NO: 73 - DNA, 447 bp, Solanum tuberosum
acaagctgcaattttgagtggtgaaggtaatgagaaggtgcaagacctgttgttgttggatgttacccctctttcccttggactggaaactgctggaggtgtcatgactgtattgatccctagaaacaccactatcccaaccaagaaagaacaggtcttctcaacctactcagacaatcagccaggtgtgttgatccaggtctatgaaggtgagcgaactagaaccagggacaacaacttgctaggcaaatttgagctctctggtattcctcctgctcccaggggagttcctcagatcacagtgtgctttgacattgatgccaatggtatcttgaatgtttctgctgaggacaagactactggacaaaagaacaagatcaccatcaccaacgacaagggcagactctccaaggaggagattgagaagatggttcaagaagcagagaa

SEQ ID NO: 74 - DNA, 632 bp, Solanum tuberosum
acaatgacataaacaacaaagcatttgtagaactatatgttaggaagggcaagttaattcggcagtttcctcaaaaatattatctatacgctgaaattgctttctttttatcatagtgttcatccaaccagcctctgatcagtttaatctcacttgatctttgatccagaagccaatcaggatcttcagcagttgcagcacgcagatctaagtgatgggcacctttcttagtagttagcgcaactatagtttccgacacatcctcaagaacactgccaccactccagggatctaacaggccatttgagaatatgatgttgctcccaaaagcttttagcgcactcttgaatgcatgtccaccaaattcagatgttatccatgtcggcctgggtttgacatgaaaatctttcaagcactactcttcattcgactttggatcataatagaattctggaaacatactagttgtcctgttacttgccataggcataatcatctcagtgcatgcctgccaatcccaaccactcataccatgaggatcatcgttcagatcgaaacagtcaactttaccggtgtaattataatagatactaattccttcaaatatacgctgcaacacactagcaccatca

SEQ ID NO: 75 - DNA, 494 bp, Solanum tuberosum
gtgagaaagatgagaaagttttggagtggagggatagtataaaacatggttgtaatccacaaaatgataacaatctttggcctcctcaaacaaggaaccaagttttgaagtatcaaaagtgggctgaaccacttgctaaaaaattgcttgaggtgctattgaagggtctcaatgtcaatgaaattgacgaatctttagaacttctcttgatgggaactatgtctataaacatcaactactatccaccatgcccaaatccaagtatcgccatgggttttcgtagacactgcgacatgggctgcataactttgctcctccaggacgacacaggaggcctatatgtccgagggactaaaggcaactggattcacgtaaatcctatcaaaggtgccttagcggttaatataggtgactcattgcagatcatgagcaatgatcgatacaagagtatcgagcattgtgtatcagttgattcgagcaggactcgaatatct

SEQ ID NO: 76 - DNA, 344 bp, Solanum tuberosum
tgatgatcctgaccgacactaaacatagtagtgttgatgggaaaaggtacaaagacgaaaattcaaaaaaataaaattgtacgcgcacgcacataaacacacatagatagatagatagatagataatagatagatcgttgatattgttcaaggcaacagggagcattcactgtatgtgaaaccactgggggacttcttcagcttacaccatgggcagaaggtaattccgatgcaatcagcgtttgtggtgcaataatcgttacaagttcccaaaatatttgagggaaatagcttcattttcagcaatgtttcttgtggtggcatgtctcggtcaggactcatca

SEQ ID NO: 77 - DNA, 278 bp, Solanum tuberosum
tagtcctgaccgacatgtgctgcaacacggcatttgggcattgaaggtttccaatattgtacaagtatacggctccggccccgaggagaggattgttaataatgtacctctcaccaatcctcagcggattgccatcttggtcataaacttcagggagtacaagattatcatcatcatgacaagttgtggggaggacaattggattctcagaagtgaaagatcggtcaggactcattgtctgcaaaggattctacccacctctcggtcaggactcatca

SEQ ID NO: 78 - DNA, 669 bp, Solanum tuberosum
tggtcagatcatgatgctttataccggtgacactgatgattatgtacaagtgcaaaatcttgcgtaccccaccaacttatctgatcctctccttctagactgggtcgagtacaaaggcaacccggttctggttcctccacccggcattggtgtcaaggactttagagacccgaccactgcttggaccggaccccaaaatgggcaatggcttttaacaatcgggtctaagattggtaaaacgggtattgcacttgtttatgaaacttccaacttcacaagctttaagctattggatgaagtgctgcatgcggttccgggtacgggtatgtgggagtgtgtggacttttacccggtatcgactgaaaaaacaaacgggttggacacatcatataacggcccgggtgtaaagcatgtgttaaaagcaagtttagatgacaataagcaagatcactatgctattgggacgtatgacttgacaaagaataaatggacacccgataacccggaattggattgtggaattgggttgaagctggattatgggaaatattatgcatcaaagacattttatgacccgaagaaacaacgaagagtactatggggatggattggggaaactgatagtgaatctgctgacctgcagaagggatgggcatctgtacagagtat

SEQ ID NO: 79 - DNA, 567 bp, Solanum tuberosum
tttttttttagggcaagaaaagaaactaatattaaccacgaatgtcaaaacaaaaagagaattataacctaacatggaactgccattctaaaagaatggcattaaaaccctatcacatattgttctgatgggataaagtaaaaactaaaaaataagtaggaagggaactcataaactatcaaaacattaataatgaacttaatcaacctcctccatcttgctgccctcagcgtcagcttcaggatcctccaatgcaggcatgtcggcatcagcatctccgctttcctcatcaatgctcaaaccgagtttcagcatcctgtgaattctgttgccaaaggtgtttggctcctcaaggctgaaacctgaggttagaagggcagtctcaaaaagcaagagaaccaagtccttcacagacttgtcattcttgtctgcatcagccctcttcctcagctcatccataatggagttctctgggttgatctccatggtcttcttgctagacatgtatccagccatgctagagtccctaagtgcctgtgccttcatgattctctccatgttagcggt

SEQ ID NO: 80 - DNA, 679 bp, Solanum tuberosum
  misc_feature (602)..(602): n is a, c, g, or t
tgaaagagcttgattccatcccttcaagcaataaacaagatcttcattatcgtccacagagagtgcgagattttcaagaacgagggagatgaagtacatgattttctcaggattagcgttgttcaattcttcatatcccctttctactgctgttctaactgtagaatcaagtgctatgtccaaaaacagcagatcctttagacggttgtttggtttgagaagcaagggcctaagctcctcacgagcctctagcaatccctcaagaagagtttccacatttttatcttccacatggtctaagacaaaatggaggaggccctgaaagccagatggcaagcctgatacaggatttatctgaactccaaccataaagccttctccctcagttttgtagcccatgcagtttgcaatagcagactcaagatctgcacctgaatgaacagccttcaatgttctcatatagtgacctaaatcacgcaaaagaccattcttttgatctcctctaaaattcggttcagaacggatagcacggtcataactcaaaagacgctcttttgttattccgttctcattcagggttttccaataaacaccaagatcanaatcactcttgatgtagtcaatcaatgcctgacagatcacaacatcatcaggactagtattattatgcaatttctga

SEQ ID NO: 81 - DNA, 609 bp, Solanum tuberosum
tgatgagtcctgaccgaaaccaattcacttgagtgagacagaggagagtggaatttcgtgtgtaggggtgaaatccggagatctacgaaggaacgccaaaagcgaaggcagctctctgggtccctaccgacgctgaggtgcgaaagcatggggagcgaacaggattagataccctggtagtccatgccgtaaacgatgagtgttcgcccttggtctacgcggatcaggggcccagctaacgcgtgaaacactccgcctggggagtacggtcgcaagaccgaaactcaaaggaattgacgggggcctgcacaagcggtggagcatgtggtttaattcgatactacgcacaaaaccttaccagcccttgacatatgaacaagaaaacctgtccttaacgggatggtacttactttcatacaggtgctgcatggctgtcgtcagctcgtgtcgtgagatgtttggtcaagtcctataacgagcgaaaccctcgttttgtgttgctgagacatgcgcctaaggagaaagtctttgcaaccgaagtgagccgaggagccgagtgacgtgccagcgctactaattgagtgccagcacctagctgcgctgtcagta

SEQ ID NO: 82 - DNA, 634 bp, Solanum tuberosum
tgatgagtcctgaccgaaaggcgccggctaccttcttactgacagcacagctaggtgctggcactcaattagtagcgctggcacgtcactcggctcctcggctcacttcggttgcaaagactttctccttaggcgcatgtctcagcaacacaaaacgagggtttcgctcgttataggacttgaccaaacatctcgcgacacgagctgacgacagccatgcagcacctgtatgaaagtaagtaccatcccgttaaggacaggtttccttgttcatatgtcaagggctggtaaggttttgcgcgtagtatcgaattaaaccacatgctccaccgcttgtgcaggcccccgtcaattcctttgagtttcggtcttgcgaccgtactccccaggcggagtgtttcacgcgttagctgggcccctgatccgcgtagaccaagggcgaacactcatcgtttacggcatggactaccagggtatctaatcctgttcgctccccatgctttcgcacctcagcgtcggtagggacccagagagctgccttcgcttttggcgttccttcgtagatctccggatttcacccctacacacgaaattccactctcctctgtctcactcaagtgaattggtttcggtc

SEQ ID NO: 83 - DNA, 375 bp, Solanum tuberosum
tgacgatgagtcctgaccgaaatggttagccataaaagttccagcaccgcactcagcattagggcactccttacgaagcctctgaacctttccagtatcatccaccttgtagaactgcaacacagcgagcttaaccttcttcttcttgtgcttgatcttctttggcttggtgtaggtcttcttcttcctcttcttagcaccaccacggagacggagcacgagatggagagttgactccttctggatgttgtagtcggcaagagtacgaccatcctcaagctgctttccggcgaaaatcaaacgctgctggtctgggggaatcccttccttgtcctggatcttggctttgacattgtcggtcaggactcatcgtca

SEQ ID NO: 84 - DNA, 317 bp, Solanum tuberosum
tgatgagtcctgaccgaaccttgcattagagaaatttcaatctccggtcttagccctttttaactctatatatttttccttgagtttcttcacctcaatttgttgtgcatgtaccatgagacgagagatctccatatcatgaatgagcattgcggttcggcactctttaaccaccatttaggacacacccgaaacgaacttactcatcttagcccttgaatctgcaaccattgttggagcatatttggacaattgagtgaactttagagcataatccctcacactcatactcccctgtcggtcaggactcatcgtca

SEQ ID NO: 85 - DNA, 239 bp, Solanum tuberosum
tgatgagtcctgaccgatcactgagatttgatgataaaccagattatgcttatctcaagagaattttccgtgaccttttcattcgtgaagggtttcagtttgattatgtatttgactggaccatattgaaatatcagcaatcacagcttgccaatattccatctcgtgctcttggcggtactgctgggccaagttcagggatacctcatggtcttgctaatgtcggtcaggactcatca

SEQ ID NO: 86 - DNA, 360 bp, Solanum tuberosum
tgatgatcctgaccgacaccttaatacaaactctaattattcatagacgcaatactttcacaaattaaagcctaaattaaaagtagcttgtcatcaaaaattaaaattaacaaaaaaaaaaaaatttattaccaaccggtctaaagcgaaacgacggttcaagcgttgattccggtcgggccgggattagtgttggatccagtacccatgtgggctacgtaaaattgtgacacgtatttgtaagctgcaccttcgggttctttcacgggtggctcatttaaatgtgcccaatggctacttatccaccatccatttttcttgattagttgatccaaactctcctgattttccaaataaaaa

SEQ ID NO: 87 - DNA, 450 bp, Solanum tuberosum
actggaaatcagaatcaaacgagcttttacccttccgttccacacgagatttctgttctcgttgagctcatcttaggacacctgcgttatcttttaacagatgtgccgccccagccaaactccccacctgacaatgtcttccgcccggatcggcccgcgaagcgagccttgggtccaaaaagaggggcagtgccccgcttccgattcacggaataagtaaaataacgttaaaagtagtggtatttcactttcgcctttcggctcccacttatactacacctctcaagtcatttcacaaagtcggactagagtcaagctcaacagggtcttctttccccgctgattctgccaagcccgttcccttggctgtggtttcgctggatagtagacagggacagtgggaatctcgttaatccattcatgcgcgtcactaattagatgacgaggcat

SEQ ID NO: 88 - DNA, 208 bp, Solanum tuberosum
gtcatactctatacatcgtgttaccacttaacatagtcctgtgtttttaataaacagtcgctaccccctggtatgtgccgctttcctaatcaaaagatgggagagcaccccttctcccgaagttacggggtcattttgccgagttccttcgacgtggttctctcaaccgccctagtatactctacttgttcacctgtgtcggtttggg

SEQ ID NO: 89 - DNA, 226 bp, Solanum tuberosum
acaagctttttttttttttttttttttttttttggaaaattaaacaattttttttttaaaagggttttttaaaccccccttttttaccaaaaaattaaaattttttttaaaaaaccttttacccccttcccccatttttgggcccacccccttttccttttcctgccccccccccaaaaaagggcccttctttttctcccatattaccccccccgcccccccttta

SEQ ID NO: 90 - DNA, 333 bp, Solanum tuberosum
tgttgacggggtatctaaggatgcccacagcaatgatagtgaagataggccaaaagaggagaaagaaacggaaggaactgatttagctatggaaaataacaatattaaccaaccagggttggttcattcacagcagcatcaatcacctgcgcctattagctgttgatgcatcatccgcatcacccttaggacatttcctgttgcatgtttactgacttttagattcaattttattattgtaataatagtaaacttcgattctctggtagtttagtgtagtttcctggactgcaatactcccatcgttatgtgtatgtatttgtttctgtcgtg

SEQ ID NO: 91 - DNA, 153 bp, Solanum tuberosum
ggccgatccgggcggaagacattgtcaggtggggagtttggctggggcggcacatctgttaaaagataacgcaggtgtcctaagatgagctcaacgagaacagaaatctcgtgtggaacagtaggttataagctgggttgattctgatttcca

SEQ ID NO: 92 - DNA, 290 bp, Solanum tuberosum
acgcttctgctggtttggacgcttctttgtaaaggcaatacagaacatcctaagagtatagctgtctgtagtctttacatcaacatgggcctcaatcaaagtctgccatttgcggaccagagacctcaacttatctgttgggaaatccattccatggaaatttgtgagaacattcctcccttgcacatcttctgctctcaaacggattttcctgaaagcctgatcctcatccttctgaagatcagccaggctaacttcaaatactctgtgttttaggccatctgaagcaa

SEQ ID NO: 93 - DNA, 443 bp, Solanum tuberosum
gagcaacttcaaatgttcaacagatatgatccattgtttatttgttcatacattttatactactacttaattcacaaatgtatttttaaccagagatttcaatagacttgacatcaagcttcttcacttgttcctttggaacagtaacagtaagcactccattctccatagaagccttaacttgatccatctttgcattctccggaagtctaaatctcctcgtgaatttcccgctgcttcgctccacgcgatgccacttatcattcttatcttctttctccacgttcctctctccgctgatttgaagaaccctatcttcttcgacttccacttttacttcctccttcttaagccctggaagatcagccttgaacacatgagcttctggagtttccttccagtctattcgcgtgttagcaaatgcagaggtctcccctgaattg

SEQ ID NO: 94 - DNA, 299 bp, Solanum tuberosum
actggaaatcagaatcaaacgagcttttacccttctgttccacacgagatttctgttctcgttgagctcatcttaggacacctgcgttatcttttaacagatgtgccgccccagccaaactccccacctgacaatgtcttgcgcccggatcggcccgcgaagcgagccttgggtccaaaaagaggggcagtgccccgcttccgattcacggaataagtaaaataacgttaaaagtagtggtatttcactttcggctttcggctcccacttatactacacctgtcaagtcatttcacaaa

SEQ ID NO: 95 - DNA, 118 bp, Solanum tuberosum
ccaaaaccataatggctgccaaaaattcacagatgaagtttgctatcttcttcgttgttctcttgacgaccactttagttaatatgtctggaatttcaaaattgcaagtgatggctat

SEQ ID NO: 96 - DNA, 100 bp, Solanum tuberosum
ccaaaaccataatggctgccaaaaattcagagatgaagtttgctatcttcttcgttgttctcttgacgaccgctttagttaatatgcaagtgatggctct

SEQ ID NO: 97 - DNA, 890 bp, Solanum tuberosum
gttttcccccgatacttggctggaactccccgtggtggcggccgctctagaactagtggatcccccgggctgcaggaattcggcacgaggcaaaacctaatggctgccaaaatttcagagatgaagtttgctatcttcttcgttgttctcttgacgaccactttagttaatatgtctggaatttcaaaattgcaagtgatggctcttcgagacatgccgccccaagaaacattgctgaaaatgaagctatttccctcaaatgttttgggaacttgtaacgattattgcaccacaaacgccgattgcttcggaattaccctctgcccgtggtgtaagctgaagaagtcccccagtggtggtacatacagtgaatgctccccgtttccttgaacaatatcaacgatctatctatctatctatgtgtgtttatgtgcgtgcgcgtacattttttttttttttgaattttcgtctttgtaccttttcccatcaacactactatatttagtgttgtctttgtatgtctttaccctttggcgtgaagaatatgaataaagggatatgtatctagatatattctaggtaatgtcctactgtttataacctatgtagcaatggttgtttgaataaaaacatactatgagtgaaataattatttcatattaaaaaaaaaaattaaaaaaaaaaaaaaaaaaaaaaaaaaactcgaggggggggcccggtaccccaattcgccctatagtgagtcgtattacaattcactggccgccgttttacaaacgtctgactgggaaaaaccctgcgttccccacttaatcgccttgcacacattccccttttgccagctggcgtaaaaaccaaaagggccgaaccgtcgccctttcccacaattg

SEQ ID NO: 98 - DNA, 224 bp, Solanum tuberosum
actggaaatcagaatcaaacgagcttttacccttctgttccacatgagatttctgttctcgttgagctcatcttaggacacctgcgttatcttttaacagatgtgccgccccagccaaactccccacctgacaatgtcttccgcccggatcggtccgcgaagcgagccttgggtccaaaaagaggggcagtgccccgcttccacctgcccgggcggccgctcga

SEQ ID NO: 99 - DNA, 452 bp, Solanum tuberosum
acccctctactggggaatcactgctgatgggtatgtggcctttgccaatgatgctgatttgcttaaaggtgcttgtggcaagtcacttgcttcttttcctcaagggtgtttctactctacaacagttggtggactgaggagctatgagaaccccaaaaacaagatcaccgccgttcctgctactgaggaagaaatatggggcgctaagtttatggtggaaggttcagctgttgttgcagccactgaatagaatatttcggttttcctctttgagctctgagggaaaaacaaggccgtgagtattagtgggggagccatgtaaataaactgaacgactactgttttctcttcctagtaattcttaagttctacttttgtgctgcttagggtagagttaggttgaaagtataatgtttggtttcttctaaggtattttaactactaaaatcaat

SEQ ID NO: 100 - DNA, 422 bp, Solanum tuberosum
accatatggaggtttctctgcagatggtggaggtggaggtggaggtgcaggtgcaggttctggtggatttgaggcccagggaacattgctcatactctgtgaatgatccacactggactgtgttgcagttggaggaagaggaggtggtggtgccatgccataataagaaggatagctgtatgaagatgttggatatgcaggttgggtatggggtggcaatgcagcgacggaattgccataggcataactgggaagcgattgaacaggagcactattatgagattgcactccgggaggataactttgttgtgtctcaccagaagatgtataattttgctggttttcgccagaagaaactgtctgagccgggacaccggaagatggtgttggcattggtggaggatactgtgcaccatatggag

SEQ ID NO: 101 - DNA, 523 bp, Solanum tuberosum
actctcaagaactccaccagcattgattaggatttgtcttgccgaagcattaatattcattgtgtcccggaaatgaggatatagaagcttatgaatagggtgaagcacacttagttgtctgtttgttgcaatcacaaatggctcgatcactgcatgagtattcaaccaatgactaattagttgatgaacaccagcgtcattcaccgcaacataagctttggccaattgccagatagagctctcaacaccttgatcacttggagtatacactttgctagtaacaccaaattgatcttcatctggatgtggcaaactcaattcaattgctagtggcttcaaagatccattatcttgtaagaagagcaaagttctcgaggcatatgatttcgttattgtagtgtttatcctcctcaaatatggtataagaagatcatgatggttcaatatgaaaagtttattattgttcatcgcctcatcaaccgttagtccatccagcttatcctctatgtgttctgcagtaatt

SEQ ID NO: 102 - DNA, 185 bp, Solanum tuberosum
tcataatctggcttctccttataatccatctgaccgatcacaacgaagatccaatgtgactggagaaccattcaataatatatgaacattacttatgagtgaattttccttctcaattatccccttagcaagctttgttggtacaccaataagttctggccagcttagttttcctttgcattgaa

SEQ ID NO: 103 - DNA, 559 bp, Solanum tuberosum
caataaaagctggggcagaaggggttcgttcagtaatattaaagggtggaggggtgctaacattggggaagatatacaacgggttagccaggagattttcagggaagatgctattggaggctgccaactatcagattagtagggaagttcttaaaaagggtggagaagtggcggctcttaacctggagtccagattggccatgctggctgcaaaaaagggcttagcaggtgctgcctcccgttattttggcttaaggagcatgatgtcatttcttggcccaattctgtggggaacacttctagcagatgttgtcattcaaatgcttggaactgattatgctagaatcgtgcgagctatttatgcatttgcacagatccgaatcacccgcacatacaaaacatctgatgttcaataaaaaagcttatttagagattggactctaaagttgcatcgtggacatcgcacactgggatattattaggctgttggctttgtatctggttctacacttgttcatagtaaatattggaaagagtgttgaagggcttgtgcttcatt

SEQ ID NO: 104 - DNA, 266 bp, Solanum tuberosum
ttcgagcgccgcccgggcaggtactcctaccattgtcattgtctgggcagttctccttgcatccatattctcattgctttgggtgcgtatcgatcctttcacatcagacgcgtcaaagactgctgcaagaggtcaatgtggtatcaactgctagtgagaagtttatttattttttaaggtaagataagatgtttgttgatccacatacggtcccatgttgtaaataagaaaactaaggcggtgtggttaagtcctgtagtcatttt

SEQ ID NO: 105 - DNA, 294 bp, Solanum tuberosum
acccatcaccatgagggacaacagcagcttcgtcgatccgacagctctagctcgtcggaggatgatggagaaggtgggaggagaaagaaggggatgaaggagaagataatggagaagatgccaggaggaggacatggccagcaggaaggtgagtatggcgctcaacatggacaaacaacaggtgaaggagctggagagaagaaaggaatgatggacaaaatcaaggacaagatccctgggatgcattgagaatacaccttaatttgctttcatttccatcttataaatgaataa

SEQ ID NO: 106 - DNA, 124 bp, Solanum tuberosum
accaagcaaactgctcgtaagtctactggaggaaaagcccccaggaagcaacttgccaccaaggctgctcgtaagtctgctcctactacaggtggagtgaagaagcctcacagataccgccctg

(19) United States
(12) Patent Application Publication
(10) Pub. No.: US 2010/0178649 A1
(43) Pub. Date: Jul. 15, 2010

(54) GENOMICS-BASED QUALITY DIAGNOSTICS FOR PREDICTION OF COLD-SWEETENNG DURING STORAGE IN PROCESSING POTATO

(75) Inventors: Monique Francisca Van Wordragen, Arnhem (NL); Renata Maria Catharina Ariens, Bergharen (NL); Peter Albert Balk, Zetten (NL); Ernst Johannes Woltering, Arnhem (NL)

Correspondence Address: FOLEY AND LARDNER LLP;SUITE 500, 3000 K STREET NW, WASHINGTON, DC, 20007, US

(73) Assignee: Agrotechnology and Food Innovations B.V.

(21) Appl. No.: 12/376,727

(22) PCT Filed: Aug. 6, 2007
(86) PCT No.: PCT/NL07/50395

§ 371 Date: Jul. 1, 2009

(30) Foreign Application Priority Data
Aug. 7, 2006 (EP) 06118520.3

(51) Int. Cl. C12Q 1/68; C07H 21/00

(52) U.S. Cl. 435/6; 536/24.3; 536/24.33

(57) ABSTRACT

The invention relates to the field of quality testing of fresh potato products. Methods, carriers and kits for determining and/or predicting the quality stage of potato batches are provided.

### FIELD OF THE INVENTION

The present invention relates to the field of quality testing of fresh plant-based potato products. Provided are methods for quality testing and quality prediction and diagnostic kits for quality screening and selection/discrimination of high, medium or low quality products or batches having approximately the same quality level. In particular, relative or absolute mRNA expression levels of defined sets of gene transcripts are determined, whereby a specific stage of a quality trait is determined. The cold-storage induced sweetening potential of batches of potato tubers can, thereby, be predicted.

### BACKGROUND OF THE INVENTION

One of the main factors influencing quality of fried potato products is potato sweetening caused by cold storage. Storage at low temperature is essential to suppress sprouting, especially when chemical sprouting inhibitors cannot be used. The cold sweetening process is caused by alterations in the starch-sugar metabolism that involves a variety of enzymes, and in addition relates to the regulation of cell osmotic.

Low environmental temperature induces the degradation of starch into sucrose. Sucrose in turn is hydrolysed into the reducing sugars glucose and fructose. During high-temperature frying the reducing sugars react with amino-acids present in the tuber (Maillard reaction), resulting in excessive browning and the development of off-flavors (Davies 1990, Am Potato J 67:815-827; Tiessen et al., 2002, Plant Cell 14: 2191-2213; Geigenberger, 2003, J of Experimental Botany 54: 475-465; Femie et al. 2002, Trends Plant Sci, 7; 35-41; Sowokinos et al. 1997, Plant Physiol. 113: 511-517).

During processing excess of soluble sugars is removed by blanching and regular replacement of satisfied washing water. The blanching process has to be adapted to the actual levels of sugar in the processed batch. For logistical reasons it is important to know in advance at what rate batches will accumulate sugars during the pre-processing storage period. At present, this is done by sampling the batches on the field, at harvest and during storage. Samples are analysed for fry colour and sometimes sugar content.

These measurements, though accurate at sampling time, do not give any information on future development of sugar content and are, therefore, of no use for in advance logistic planning (F. P. Scheer and J. Wijnen, 2005, Pieperprofijt. Openbare eindrapportage AKK-project code ACD-03.031).

Mismatches between predicted and actual quality are very costly to the industry for three reasons:
low quality product has to be sold on low-price markets
severe mismatches require re-programming of the production process (additional washing steps) which is time consuming and therefore expensive
batches with unacceptable low quality have to discarded.

It is, therefore, an object of the invention to provide an easy method to assess and predict the quality of potato batches, especially of processing potatoes. These tests have a high information content, allowing prediction of the future batch quality with respect of cold-storage induced sweetening and can thus be used as support tool, for decisions concerning applications, treatments or destinations of specific potato batches. In addition, the optimal time of harvest can be determined.

### GENERAL DEFINITIONS

The term “gene” means a DNA fragment comprising a region (transcribed region), which is transcribed into an RNA molecule (e.g. an mRNA, or RNA transcript) in a cell, operably linked to suitable regulatory regions (e.g. a promoter). A gene may thus comprise several operably linked fragments, such as a promoter, a 5′ leader sequence, a coding region and a 3′nontranslated sequence comprising a polyadenylation site.

“Indicator genes” refers herein to genes whose expression level is indicative of a certain quality stage of a fresh agricultural product, especially sweetening stage and sweetening potential of batches of potato tubers.

“Expression of a gene” refers to the process wherein a DNA region which is operably linked to appropriate regulatory regions, particularly a promoter, is transcribed into an RNA molecule.

“Upregulation” of gene expression refers to an amount of mRNA transcript levels of at least about 2 times the level of the reference sample, preferably at least about 3×, 4×, 5×, 10×, 20×, 30× or more.

“Downregulation” of gene expression refers to an amount of mRNA transcript levels of at least about 2 times lower than the level of the reference sample, preferably at least about 3×, 4×, 5×, 10×, 15× lower.

“Constant” refers to an essentially equivalent mRNA transcript level as in the reference sample. Generally, housekeeping genes (such as glyceraldehydes-3-phosphate dehydrogenase, albumin, actins, tubulins, 18S or 28S rRNA) have a constant transcript level.

“Relative” mRNA expression levels refer to the change in expression level of one or more indicator genes relative to that in another sample, preferably compared after “normalization” of the expression levels using e.g. housekeeping genes or other reference genes. The fold change (upregulation or downregulation) can be measured using for example quantitative real-time PCR. The fold change can be calculated by determining the ratio of an indicator mRNA in one sample relative to the other. Mathematical methods such as the 2(-Delta Delta C(T)) method (Livak and Schmittgen, Method 2001, 25: 402-408) or other mathematical methods, such as described in Pfaffl (2001, Nucleic Acid Research 29: 2002-2007) or Peirson et al. (2003, Nucleic Acid Research 31: 2-7) may be used.

“Absolute” mRNA expression levels refer to the absolute quantity of mRNA in a sample, which requires an internal or external calibration curve and is generally more time consuming to establish than relative quantification approaches.

The term “training sample” or “training batch” refers herein to a reference batch of the same type of plant material (e.g. same tissue type and cultivar), having a predetermined quality status (i.e. the expression profile of indicator genes of the training batch is correlated with the quality stage or predicted/future quality stage). The expression profile of the indicator genes in a “test batch” can then be analyzed and thereby correlated with the quality stage (or predicted/future quality stage) of one of the training batches.

The term “substantially identical”, “substantial identity” or “essentially similar” or “essential similarity” means that two peptide or two nucleotide sequences, when optimally aligned, such as by the programs GAP or BESTFIT using default parameters, share at least a certain percent sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear that when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. or using in EmbossWIN (version 2.10.0) the program “needle”, using the same GAP parameters as described above. For comparing sequence identity between sequences of dissimilar lengths, it is preferred that local alignment algorithms are used, such as the Smith Waterman algorithm (Smith T F, Waterman M S (1981) J. Mol. Biol 147(1); 195-7), used e.g. in the EmbossWlN program “water”. Default parameters are gap opening penalty 10.0 and gap extension penalty 0.5, using Blosum62 for proteins and DNAFULL matrices for nucleic acids.

“Stringent hybridization conditions” can also be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100 nt) are for example those which include at least one wash in 0.2×SSC at 63° C. for 20 min, or equivalent conditions. Stringent conditions for DNA-DNA hybridization (Southern blots using a probe of e.g. 100 nt) are for example those which include at least one wash (usually 2) in 0.2×SSC at a temperature of at least 50° C., usually about 55° C., for 20 min, or equivalent conditions.

The term “comprising” is to be interpreted as specifying the presence of the stated parts, steps or components, but does not exclude the presence of one or more additional parts, steps or components. A nucleic acid sequence comprising region X, may thus comprise additional regions, i.e. region X may be embedded in a larger nucleic acid region.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The term “plant” refers to any organism of which the cells, or some of the cells contain chloroplasts. It may refer to the whole plant (e.g. the whole seedling) or to parts of a plant, such as cells, tissue or organs (e.g. pollen, seeds, gametes, roots, leaves, flowers, flower buds, anthers, fruit, tubers, etc.) obtainable from the plant, as well as derivatives of any of these and progeny derived from such a plant by selfing or crossing. “Plant cell(s)” include protoplasts, gametes, suspension cultures, microspores, pollen grains, etc., either in isolation or within a tissue, organ or organism.

The term “batch” refers to a collection of harvested plant products (especially harvested potato tubers) that share a considerable part of their history in the production and or distribution chain. For example the term “batch” is used to describe a group of plant products grown in the field in the same period and harvested at the same time. Potato tubers in a batch may be of the same variety or a mixture of different varieties.

The term “quality trait” refers to a specific physiological characteristic of a plant product that is important for determining the economic value. Herein, the quality trait which is assessed and predicted is sweetening level and sweetening potential of potato tubers, especially of batches of pre-processing potato tubers stored under cold-storage conditions.

The term “quality stage” or “quality trait stage” refers to a predefined moment in the development of a quality trait, described by a specific set of physiological of morphological characteristics. For example, the term is used herein to refer to a specific level sugar accumulated in potato tubers, as determined e.g. by fry color and/or sugar content analysis.

“Predicted or future quality (trait) stage” is the quality stage which is predicted to develop in the batch after time, as determined by the expression profile of a set of indicator genes. This is encompassed by the broader term “quality stage”.

The term “fresh” refers to plant products that have not (yet) been processed, or only minimally processed (e.g. cut or sliced and/or packaged) after harvest and which are still actively metabolizing and responsive to the environment.

“PCR primers” include both degenerate primers and non-degenerate primers (i.e. of identical nucleic acid sequence as the target sequence to which they hybridize).

“Oligonucleotides” refer to nucleic acid fragments suitable for use as PCR primers or hybridization probes, e.g. coupled to a carrier in a nucleic acid microarray.

“DNA Microarray” or “DNA chip” is a series of known DNA sequences (oligonucleotides or oligonucleotide probes) attached in a regular pattern on a solid surface, such as a glass slide, and to which a composition consisting of or comprising target sequences are hybridized for identification and/or quantification.

### DETAILED DESCRIPTION

A genomics-based method is provided herein that can be used for measuring and predicting specific quality characteristics (or quality stage) of fresh potato products. The tests are based on the combined expression profiles of a carefully selected set of indicator genes.

In living organisms, each developmental step and every interaction with the environment is orchestrated by DNA encoded genes. The history and actual condition of a plant, animal or microorganism is accurately reflected in the activity profile of its genes. The indicators are selected by combining gene expression analysis (using microarrays) and thorough physiological analyses with knowledge of distribution chain logistics. The information is used to select those genes that are most involved in determining cold sweetening potential. A subset of this selection of indicator genes is translated into a reliable and robust assay for predicting cold sweetening stage and cold sweetening potential of potato batches.

Quality assays and kits are provided for the determination/prediction of potato sweetening during cold storage. A set of genomics-based indicators is described that can be used in a quality test for predicting, e.g. at harvest time or post-harvest, the future rate of sugar accumulation during cold storage. In addition the test can be used to identify batches of harvested potato tubers which have a similar present or predicted level of sugar accumulation. The test is based on the combined expression profiles of a carefully selected set of indicator genes.

In its broadest term, the method for determining the quality stage and/or predicting quality traits according to the invention comprises:
(a) providing a nucleic acid sample (comprising RNA or corresponding cDNA) of a potato plant or plant part, or a plurality of plants (batch), especially of potato tubers or parts thereof,
(b) analyzing the sample by determining the level of a set of indicator mRNA transcripts in the sample, which are indicative of the present stage and/or of the future (predicted) status of a quality trait of the plant or plant part, or the batch of plants (especially the cold sweetening level and potential, i.e. future rate of sweetening), and optionally
(c) identifying and selecting the plant or the batch, which comprises a certain level of the indicator mRNA transcripts (i.e. a certain relative or absolute amount of mRNA or corresponding cDNA) for further use.

In one embodiment steps (a) and (b) are repeated at regular time intervals, until the mRNA transcript levels are such that the plants, plant parts or batch is at the right quality stage for step (c).

Preferably the plants or plant parts (or batches) are harvested parts, most preferably harvested potato tubers or parts thereof. The quality stage of the harvested product is determined one or more times, for example at harvest and/or one or more times after harvest. A harvested product may also refer to harvested plants or plant parts which have been further processed, such as sliced, diced, etc. and packaged into batches, but which are preferably still regarded as “fresh” (as defined above).

In one embodiment, the expression of the indicator genes is carried out after a short cold-shock treatment, as described in the Examples, e.g. about 24 hours (1 day) at about 2° C., because the correlation between expression levels of the indicator genes and the quality prediction of the batch was found to be very robust using this method. Thus, optionally prior to step (a) a cold-treatment may be applied to the harvested plant material. For example, expression levels of indicator genes before cold treatment (e.g. at harvest/intake) and after cold-treatment may then be compared in order to determine the cold-sweetening potential of one or more batches (see Examples). Alternatively, the relative or absolute mRNA level of the indicator genes after cold-treatment is compared to the level of the indicator RNAs in one or more suitable reference samples.

In one embodiment steps (a) and (b) of the method are carried out at regular time intervals (e.g. once a week, once every two weeks, once a month, once every two or three months, etc), so that a change in the level of mRNA transcripts (or the corresponding cDNAs) of the indicator genes can be determined. The relative change in mRNA transcript abundance (up-regulation down-regulation, no change in mRNA levels) is then used to select plants, plant parts or batches in step (c). Alternatively, the relative or absolute mRNA level of the indicator genes is compared to the level of the indicator RNAs in one or more suitable reference samples. Such a control may for example consist of one or more nucleic acid samples of known quality stages (e.g. training batches or batches obtained at earlier time points) so that the indicator mRNA abundance is compared relative to that of the reference sample(s). It is understood that the control expression data does not need to be produced at the same time as the sample data, but can have been produced previously, such as one or more training batches.

The nucleic acid sample of step (a) may be provided for several individual plants (or parts), or preferably for batches of several plants (or parts), especially for batches of harvested potato tubers (or parts thereof). cDNA samples of batches may be made by either first pooling tissue from several individuals (e.g. from at least about 5 or 10 potato tubers) and then obtaining the nucleic acid from the pooled tissue sample or by directly pooling the nucleic acid obtained from individual potato tubers. Preferably, the nucleic acid sample in step (a) comprises or consists of total RNA, total mRNA or total cDNA. For example, the total mRNA is isolated (e.g. using polyA⁺ selection) and is used to make corresponding cDNA by reverse transcription using known methods.

The mRNA level (or corresponding cDNA level) of a set of defined indicator genes can be detected and quantified using various methods generally known in the art, such as (but not limited to) quantitative PCR methods, preferably quantitative RT-PCR, or nucleic acid hybridization based methods (for example microarray hybridization). Quantitative PCR (qPCR) may be carried out by conventional techniques and equipment, well known to the skilled person, described for instance in S. A. Bustin (Ed.), et al., A-Z of Quantitative PCR, IUL Biotechnology series, no 5, 2005. Preferably, labeled primers or oligonucleotides are used to quantify the amount of reaction product. Other techniques capable of quantifying relative and absolute amounts of mRNA in a sample, such as NASBA (Nucleic Acid Sequence Based Amplification), may also be suitably applied. A convenient system for quantification is the immuno labeling of the primers, followed by an immuno-lateral flow system (NALFIA) on a pre-made strip (references: Kozwich et al., 2000, Applied and Environmental Microbiology 66, 2711-2717; Koets et al., 2003, In: Proceedings EURO FOOD CHEM XII—Strategies for Safe Food, 24-26 Sep. 2003, Brugge, Belgium, pages 121-124; and van Amerongen et al., 2005 In: Rapid methods for biological and chemical contaminants in food and feed. Eds. A. van Amerongen, D. Barug and M. Lauwaars, Wageningen Academic Publishers, Wageningen, The Netherlands, ISBN: 9076998531, pages 105-126).

As a positive control for the RNA isolation, reverse transcriptase reaction, amplification reaction and detection step, amplification and detection of a constitutively expressed housekeeping gene may be included in the assay, such as ribosomal (18S or 25S) rRNA's, actin, tubulin or GAPDH. Primers may be labeled with direct labels such as FITC (fluorescein), Texas Red, Rhodamine and others or with tags such as biotin, lexA or digoxigenin which may be visualized by a secondary reaction with a labeled streptavidin molecule (for instance with carbon or a fluorescent label) or a labeled antibody (labeled with fluorescent molecules, enzymes, carbon, heavy metals, radioactive isotopes or with any other label).

In another embodiment, comparative hybridization is performed on mRNA or cDNA populations obtained from a plant or sample thereof, to a set of indicator gene sequences, which may optionally be tagged or labeled for detection purposes, or may be attached to a solid carrier such as a DNA array or microarray. Suitable methods for microarray detection and quantification are well described in the art and may for instance be found in: Applications of DNA Microarrays in Biology. R. B. Stoughton (2005) Annu. Rev. Biochem. 74:53-82, or in David Bowtell and Joseph Sambrook, DNA Microarrays: A Molecular Cloning Manual, Cold Spring Harbor Laboratory Press, 2003 ISBN 0-870969-625-7. To construct a DNA microarray, nucleic acid molecules (e.g. single stranded oligonucleotides according to the invention) are attached to a solid support at known locations or “addresses”. The arrayed nucleic acid molecules are complementary to the indicator nucleotide sequences according to the invention, and the location of each nucleic acid on the chip is known. Such DNA chips or microarrays, have been generally described in the art, for example, in U.S. Pat. No. 5,143,854, U.S. Pat. No. 5,445,934, U.S. Pat. No. 5,744,305, U.S. Pat. No. 5,677,195, U.S. Pat. No. 6,040,193, U.S. Pat. No. 5,424,186, U.S. Pat. No. 6,329,143, and U.S. Pat. No. 6,309,831 and Fodor et al. (1991) Science 251: 767-77, each incorporated by reference. See also technology providers, such as Affymetrix Inc. (www.affymetrix.com). These arrays may, for example, be produced using mechanical synthesis methods or light-directed synthesis methods that incorporate a combination of photolithographic methods and solid phase synthesis methods. Also methods for generating labeled polynucleotides and for hybridizing them to DNA microarrays are well known in the art. See, for example, US 2002/0144307 and Ausubel et al., eds. (1994) Current Protocols in Molecular Biology, Current Protocols (Greene Publishing Associates, Inc., and John Wiley & Sons, Inc., New York; 1994 Supplement).

Herein a specific “set of indicator genes” is provided, whose expression level correlates with and is indicative of the present and/or future sweetening stage and sweetening potential of the plant, plant part or preferably the batch. A “set of indicator genes” refers, therefore, to a defined number of genes whose expression level (mRNA abundance, or corresponding cDNA abundance) is being determined. A distinction can be made between the “main set”, which refers to a larger number of defined genes, and “sub-sets”, which refer to smaller numbers selected from the main set. Thus, either the main set of indicator transcripts may be detected or, preferably, a subset is detected. For example, the upregulation of one indicator mRNA transcript and the down regulation of another indicator mRNA transcript may already be sufficient to determine the quality of the batch. Thus, although 106 indicator genes (and indicator transcripts) are provided herein, any subset thereof, such as 100, 99, 96, 94, 90, 86, 74, 60, 50, 32, 30, 25, 20, 15, 10, 5, 4, 3, or 2 may already be sufficient. These 106 indicator genes, or any subset of these, may be used with or without a cold treatment step. Especially, 20 indicator genes (SEQ ID NO: 1-20) or a subset thereof is preferably used in a method without cold treatment, and/or 86 indicator genes (SEQ ID NO: 21-106) or a subset thereof is preferably (but not solely) used with a cold-treatment step included. In addition it is clear, that the robustness of the method is inversely related to the number of indicator transcripts being detected. A lower number of indicator transcripts being detected may need to be compensated by a larger number of samples analyzed.

When referring to “indicator genes”, not only the specific nucleic acid sequences (mRNA or cDNA) of those genes (as depicted in the Sequence Listing) are referred to, but also “variants” of these sequences and fragments of the indicator genes or of the variants. A “variant” refers herein to a nucleic acid sequence which are “essentially identical” to the indicator genes provided, i.e. they comprises at least about 70, 75, 80, 85, 90, 95, 98, 99% or more, nucleic acid sequence identity to the sequences provided herein (determined using pairwise alignment with the Needleman and Wunsch algorithm, or with the Smith Waterman algorithm, as defined).

As mentioned, also fragments (e.g. oligonucleotides) of indicator genes (or of the variants of indicator genes) are encompassed and may be detected, or may be used for detection and quantification of the indicator transcript in a sample or batch. Fragments comprise any contiguous stretch of at least 8, 10, 11, 12, 13, 14, 15, 16, 17, 18, 19, 20, 21, 22, 23, 24, 25, 30, 40, 50, 100, 200, 500, 800, 900, 1000 or more nucleotides of an indicator gene or a variant thereof. Such fragments may be used as PCR primers or probes for detecting indicator genes by selectively hybridizing to the indicator mRNA or cDNA.

Variants may be isolated from natural sources (e.g. other Solanum tuberosum varieties, breeding lines or accessions), using for example stringent hybridization conditions or can be easily generated using methods known in the art, such as but not limited to nucleotide substitutions or deletions, de novo chemical synthesis of nucleic acid molecules or mutagenesis- or gene-shuffling techniques, etc.

Also provided are kits for carrying out the methods and nucleic acid carriers comprising sets of indicator genes, and/or variants and/or fragments of indicator genes, e.g. oligonucleotides of the indicator genes or of variants thereof.

Nucleic acid carriers may for example be arrays and microarrays or DNA chips, comprising nucleotides on a glass, plastics, nitrocellulose or nylon sheets, silicon or any other solid surface, which are well known in the art and for instance described in Bowtell and Sambrook, 2003 (supra) and in Ausubel et al., Current protocols in Molecular Biology, Wiley Interscience, 2004. A carrier according to the current invention comprises at least two (or more, such as at least 5, 10, 15, 20, 25, 30, 40, 50, 60, 70, 74, 80, 90, 94, 96, 97, 99, 100 or more such as 101, 102, 103, 104, 105 or 106) (oligo-)nucleotide probes capable of selectively hybridizing with at least the two (or more) indicator genes (mRNA or cDNA) present in a sample.

A kit for determining and/or predicting the quality stage of a sample comprises elements for use in the methods of the invention. Such a kit may comprise a carrier to receive therein one or more containers, such as tubes or vials. The kit may further comprise unlabeled or labeled (oligo)nucleotide sequences of the invention, e.g. to be used as primers, probes, which may be contained in one or more of the containers, or present on a carrier. The (oligo)nucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labeled probes), nucleoside triphosphates, filter paper, gel materials, transfer materials, electrophoresis materials and visualization materials (preferably dyes, labeled antibodies or -enzymes) autoradiography supplies. Such other components for the kits of the invention are known per se. The kit may also comprise tissue samples and/or nucleic acid samples, such as suitable control samples.

Assays and Kits for the Determination and/or Predicting Cold-Storage Induced Sweetening and/or Sweetening Potential in Potato Tubers

In one embodiment of the invention a method for determining and/or predicting cold-storage induced sweetening and/or sweetening potential, in potato tubers is provided.

As mentioned above, harvested potato tuber batches accumulate sugars during cold-storage at about 4° C. The present method can be used to a) differentiate between the present sweetening stages of batches and, more importantly, b) differentiate between batches having a different potential for sweetening during future storage.

The method provided herein uses a set of 106 indicator genes (a set of 20 and/or a set of 86 genes), or a subset thereof, whose expression profile can be used as measurement for the sweetening level of potato tubers and for predicting the future sweetening potential of a batch. Thus, based on the relative or absolute expression level of the described indicator genes conclusions can be drawn about the level of sweetening and/or the level of sweetening potential of potato tuber batches in storage. This way batches which are likely to develop high levels, medium levels or low levels of sweetening during storage can be discriminated quickly.

One additional advantage is that batches can be identified which develop reduced or low levels of acrylamide after frying. When potatoes are fried, the reducing sugars together with asparagines result in the formation of the carcinogen acrylamide. The present test can therefore be used to identify batches having a low sweetening level and/or low sweetening potential, for the manufacture of fried potato products comprising low acrylamide levels.

It was found that the selected set of 106 (a set of 20 and/or of 86) indicator genes, and subsets thereof, can be used as a diagnostic tool to predict cold sweetening potential of potatoes for at least 3 months and probably longer, since in general sugar-starch metabolism is most active in the first 3 months of storage. The indicators can be used to assist storage planning according to the FEFO principle (first expired, first out) since it allows identification of high risk batches that should be processed first. Low risk batches can be stored for longer.

In addition the test can be used for processing planning for the frying industry. Batches with comparable sugar accumulating potential can be sorted out and processed in one string. This will reduce the number of shifts in processing parameters and will, therefore, enhance the cost-efficiency.

The method for determining the sweetening level and/or the sweetening potential of potato tuber batches comprises the following steps:
(a) providing a nucleic acid sample (comprising mRNA or cDNA) of a batch of potato tubers (e.g. a representative sample of tubers or tuber parts),
(b) analysing the sample by determining the level of a set of indicator mRNA transcripts in the sample, which are indicative of the sweetening stage and/or sweetening potential of the batch, and optionally
(c) identifying and selecting the potato tuber batch which comprises a certain level of the indicator mRNA transcripts, relative to suitable controls, for further use, e.g. for immediate processing or for further cold storage. Thus, batches which comprise an “indicator mRNA profile” which is indicative of a certain sweetening level or sweetening potential are identified.

For example, the following types of batches can be distinguished using the indicator genes:
a) Top quality batches, having a low potential for accumulating sugars during cold storage and, thus, having a low potential for sweetening during cold-storage; these batches only develop between about 0.10 and 0.30 gram glucose/100 g dry weight when stored at 4° C. and only between about 0.01 and 0.06 gram glucose/100 g dry weight when stored at 8° C. As a consequence they can be kept in cold-storage for prolonged periods of time, such as at least three months, but likely 4, 5 or 6 months, or more.
b) Medium quality batches having a medium potential for accumulating sugars and for sweetening during cold-storage; these batches develop between about 0.30 and 0.50 gram glucose/100 g dry weight when stored at 4° C. and between about 0.06 and 0.10 gram glucose/100 g dry weight when stored at 8° C.
c) Low quality batches having a high potential for accumulating sugars and for sweetening during cold-storage; these batches develop between about 0.50 and 1.00 gram glucose/100 g dry weight when stored at 4° C. and between about 0.10 and 0.20 gram glucose/100 g dry weight when stored at 8° C. Preferably, their cold-storage time is therefore reduced to less than three months, such as only days or weeks.

The method can be applied to any potato plants/tubers of the genus “Solanum tuberosum”, including ‘consumption varieties’, starch or seed potatoes, transgenic plants, and the like. Preferably, nucleic acids of a batch of tubers refers to nucleic acids obtained from a batch of plants grown at the same location and under the same growth conditions. Preferably, but not necessarily, a batch is composed of the same plant variety or line. In one embodiment the batches are composed of tubers of the variety Agria and/or Bintje, although also other varieties may be used.

The method can be used to identify and select those tuber batches which have a high, medium or low sweetening potential when stored for several weeks or months (e.g. 1-3 months, or longer, such as 4, 5 or 6 months) under cold storage. Thus, batches having an identical sweetening stage or identical sweetening potential can be identified and selected for further cold storage or for immediate further use (e.g. processing).

Cold storage refers to storage for several weeks or months in controlled environments at temperatures from approximately +4° C. to +8° C. Cold shock or cold treatment refers to short term storage (up to 36 hrs) of a sample taken from a batch potatoes, at a temperature of about +2° C. This is used as a means to enhance the physiological differences between batches for more easy and robust determination

Preferably, the RNA profile of the 106 indicator genes, or of a subset thereof (e.g. the indicator genes of SEQ ID NO: 1-20 and/or the 86 indicator genes of SEQ ID NO: 21-106, or variants thereof, or a subset of any of these), is analyzed more then once, i.e. at one or more time interval's. This allows the expression level of the indicator genes to be compared relative to the earlier level(s). For example, once a month, once every 3, 2, or 1 week, the mRNA profiling method may be repeated. Alternatively, expression levels of a test batch are compared to the expression levels of one or more training batches (for example a batch of tubers having a known sweetening potential). As mentioned, in one embodiment, a prior cold-shock step of the batches/samples is included, as expression profiles show a robust correlation with the predicted cold sweetening potential in this method. In this method preferably a set of 86 indicator genes or a subset thereof is used (SEQ ID NO: 21-106, and variants thereof).

Any part of the potato tuber(s) may be used to prepare the nucleic acid sample. Thus, first suitable tissue is sampled for nucleic acid extraction. In the present method, it is preferred that in step (a) a nucleic acid sample is prepared by obtaining tissue from a representative number of tubers (e.g. at least about 5, 6, 7, 8, 9, 10, 15, 20 or more tubers of a batch) and extracting the total RNA or total mRNA from the pooled sample. The sample can be prepared using known nucleic acid extraction methods, e.g. total RNA or mRNA purification methods and kits provided in the art (e.g. RNAeasy kits of Qiagen, kits of BIORAD, Clontech, Dynal etc.). The mRNA may be reverse transcribed into cDNA, using known methods.

In step (b), the nucleic acid sample is analysed for the presence and the level (abundance or relative level) of indicator RNA transcripts (mRNA) in the sample. When referring to indicator RNA in a sample, it is clear that this also encompasses indicator cDNA obtainable from said mRNA.

In one embodiment, the mRNA (or cDNA) sequences indicative of sweetening level and/or sweetening potential which are detected in the sample are SEQ ID NO: 1-106 (i.e. SEQ ID NO: 1-20 and/or SEQ ID NO: 21-106), or variants thereof, or fragments of any of these (the main set of indicator genes). Thus, any method may be used to detect the relative or absolute amounts of SEQ ID NO: 1-106, variants of SEQ ID NO: 1-106, or fragments of these in the sample(s). For example, PCR primer pairs which amplify fragments of each of SEQ ID NO: 1-106 may be used in quantitative RT-PCR reactions. Alternatively, the nucleic acid sample may be labeled and hybridized to a nucleic acid carrier comprising oligonucleotides of each of SEQ ID NO: 1-106, whereby the level of these transcripts in the sample is determined.

In another embodiment a subset of indicator genes is detected in the sample, and the transcript level is compared to the transcript level of the same subset in a suitable control. A subset may be chosen from SEQ ID NO: 1-106 (or variants thereof), or preferably from SEQ ID NO: 1-20 (or variants thereof) or from SEQ ID NO: 21-106 (or variants thereof). Examples of suitable subsets include SEQ ID NO: 21-94, SEQ ID NO: 1-94, any 2, 3, 4, 5 (or more) sequences selected from SEQ ID NO: 1-20 or 1-94 or 21-94 or 95-106 or 1-106 or 21-106. Other suitable subsets are subsets comprising or consisting of at least about 2, 3, 4, 5, 10, 15 or more of SEQ ID NO: 10, 24, 25, 30, 43, 48, 66, 69, 73, 76, 78, 79, 83, 86, 87, 89, 90, 92, 95, and 98 (and/or variants of any of these) and/or of SEQ ID NO: 78, 86, 89, 95, 96, 97, 98, 99, 100, 101, 102, 103, 104, 105, and 106 (and/or variants of any of these).

Also, SEQ ID NO: 1-9, and variants thereof, are highly expressed in high sugar accumulators at harvest/intake and thus in batches having a high sweetening potential (and poor quality). SEQ ID NO: 10-20, and variants thereof, are highly expressed in low sugar accumulators at harvest and thus in batches having a low sweetening potential (and high/top quality, see Table 1). Highly expressed is herein defined as upregulated compared to a control transcript, in which the fold change between high and low expression is at least 2, but averages between 10- and 30-fold. The control transcript can be any household gene transcript, or a combination of these, that is/are expressed at constant levels under all circumstances, for example actins, histones, glyceraldehyde-3-phosphate dehydrogenase (GAPDH), 18S rRNA and/or ubiquitin. 18s rRNA is the most preferred reference transcript for determining high and/or low expression of transcripts chosen from SEQ ID NO's 1-106.

Thus, a high expression level of SEQ ID NO: 1-9 and/or a low expression level of SEQ ID NO: 10-20 at harvest correlates with a high quality of a batch (i.e. a low sweetening potential) and can be used to discriminate batches. Thus, SEQ ID NO: 1-20, or variants thereof, or a subset thereof, can be used in a quick quality prediction assay at about harvest, whereby the test is performed on samples only once. This quick assay can be used to sort batches for further use, prior to storage.

Most preferably, the mRNA or cDNA level of a set of at least 2, 3, 4, 5, 6, 7, 8, 9, 10, 15, 16, 17, 18 or 19 or more of any one of SEQ ID NO: 1-20, or variants or fragments thereof, is determined in the sample in step (b).

Alternatively or in addition mRNA or cDNA levels of SEQ ID NO: 21-106 (or variants or fragments thereof), or any subset thereof, such as 2, 3, 5, 10, 20, 30, 40, 50, 60, 70, 80, 85, or more, is determined, optionally after a one day cold-shock. The expression level of the indicator transcripts is preferably compared to the level transcripts of a suitable control, e.g. a sample of the same batch that was not cold treated, or compared to a sample of a batch having a known high, medium and/or low sweetening potential.

In a preferred embodiment the expression level of at least one transcript of SEQ ID NO: 1-9 and at least one transcript of SEQ ID NO: 10-20 are used as indicator transcript. In another preferred embodiment at least 2, or 3 transcript of SEQ ID NO: 1-9 and at least 2 or 3 transcript of SEQ ID NO: 10-20 are used as indicator transcript. Thus, the “minimal set” of indicator mRNAs comprises two mRNAs, at least one selected from SEQ ID NO: 1-9 (or variants or fragments thereof) and at least one selected from SEQ ID NO: 10-20 (or variants or fragments thereof).

As already mentioned, it is understood that also “variants” of SEQ ID NO: 1-106 may be detected in a sample, such as nucleic acid sequences essentially similar to any of SEQ ID NO: 1-106, i.e. comprising at least 70, 75, 80, 85, 90, 95, 98, 99% or more nucleic acid sequence identity to any of SEQ ID NO: 1-106. Such variants may for example be present in different potato species or different potato varieties or breeding material.

The actual method used for determining the level of the set of indicator mRNA transcripts is not important. Any gene expression profiling method may be used, such as RT-PCR, microarrays or chips, Northern blot analysis, cDNA-AFLP, etc. See elsewhere herein. For example, PCR primer pairs for each of SEQ ID NO: 1-106 may be designed using known methods. In one embodiment of the invention two or more of these primer pairs are used in the method. Alternatively, nucleic acid probes, which hybridize to SEQ ID NO: 1-106 may be made for use in the detection. Any fragment of 15, 20, 22, 30, 50, 100, 200, 300, 500 or more consecutive nucleotides of SEQ ID NO: 1-106, or the complement strand, or of a variant of SEQ ID NO: 1-106, may be suitable for detection of the full length transcript in a sample. Equally, any fragment of a “variant” of any one of SEQ ID NO: 1-106 (as defined above) may be used.

In one embodiment a carrier is provided comprising nucleic acid molecules SEQ ID NO: 1-106, variants of SEQ ID NO: 1-106 and/or most preferably fragments (oligonucleotides) of any of these or of a subset of any of these. The carrier may, for example, be contacted under hybridizing conditions with the (labeled) nucleic acid sample of the sample of step (a), allowing detection of the level of each of the indicator transcripts present in the sample.

If the expression profile of the indicator mRNAs of the test batch corresponds to the profile of a reference batch having a certain sweetening potential, the batch can be identified and selected for further use.

In a further embodiment, kits, oligonucleotides (e.g. PCR primers, nucleic acid probes) and antibodies are provided, for determining the sweetening potential of potato tuber batches. Such kits comprise instructions for use and one or more reagents for use in the method. Optionally, tissue samples or nucleic acid samples suitable as controls may be included. Thus, such a kit may comprise a carrier to receive therein one or more containers, such as tubes or vials. The kit may further comprise unlabeled or labelled oligonucleotide sequences of the invention (SEQ ID NO: 1-106, or variants thereof, or parts thereof, such as degenerate primers or probes), e.g. to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labelled probes), nucleoside triphosphates, filter paper, gel materials, transfer materials, electrophoresis materials and visualization materials (preferably dyes, labelled antibodies or -enzymes) autoradiography supplies.

### FIGURE LEGENDS

FIG. 1 (A, B, C, D and E): Glucose Accumulation during potato tuber storage

A: absolute levels at intake and after 3 months at 4° C. or 8° C. storage.
B: lines sorted according to glucose increment between intake and 3 months storage at 4° C. The fry colour is depicted for comparison. The 5 least accumulating and the 5 most accumulating lines were selected for micro-array analysis.
C: Seasonal effect. Comparison of the frying colour at intake of the various lines in the two subsequent years of the trials.
D: Sugar build up during storage of 6 different Bintje and Agria batches in experiment started September 2005. About 400 to 450 tubers were used at each sampling date, and a sample of 20 tubers was taken from each treatment.
E: Sugar build up during storage of 6 different Agria batches (designated V18, V19, V23, V24, V25 and V27) in experiment started September 2006. About 350 to 400 tubers were used at each sampling date, and a sample of 20 tubers was taken from each treatment.

FIG. 2 A, B and C: Predictive value of indicator subsets on strong or weak accumulating potato lines or batches, using PAM analysis.

Diamonds indicates: predicted as strong; squares indicates: predicted as weak. Predictions were done at the intake for storage. Determination of accumulation potential was done after three months and compared with the prediction.

A: containing 2 indicators tested on different lines and cultivars.
B: containing 5 indicators tested on different lines and cultivars.
C: Containing 30 indicators tested on different high and low sugar accumulator batches (origins) of Agria selected from two seasons.

FIG. 3:

PCA plots based on gene expression profiles before long term storage on batches Bintje and Agria from different growers. The plots demonstrate that the selected indicator genes (among them the sequence having SEQ ID NO: 10, 24, 25, 30, 43, 48, 66, 69, 73, 76, 78, 79, 83, 86, 87, 89, 90, 92, 95 and 98) are able to discriminate between batches before storage. The groups are in agreement with the quality classes designated after storage on the basis of sugars content and frying colour.

A: Bintje start samples 2005
B: Agria cold-induced samples 2005
C: Agria cold induced samples 2006

### SEQUENCES

SEQ ID NO 1-20: Twenty potato indicator genes, especially (but not necessarily) for use at intake/harvest and without a cold treatment period prior to expression analysis.

SEQ ID NO: 21-106: Eighty-six potato indicator genes, especially (but not necessarily) for use in combination with a cold treatment period prior to expression analysis.

### EXAMPLES

### Example 1

### Cold-Storage Induced Sweetening Potential in Potato Tubers

### 1.1. Material and Methods

### 1.1.1 Species and Plant Material

The method was developed and validated for Solanum tuberosum and focused on so-called ‘consumption varieties’ but may also be applied to starch or seed potatoes. Experiments were performed using the diploid, segregating population RH94-076 (Tae-Ho Park, 2005, Identification, characterization and high-resolution mapping of resistance genes to Phytophthora infestans in potato. Wageningen Dissertation no. 3745. Chapter 2) kindly provided by Prof. dr. R. Visser, Wageningen University and a series of 10 commercial lines, namely: Agria, Astarte, Bintje, Gloria, Granola, Innovator, Karnico, Nicola, Premiere and Saturna. Seed potatoes were planted in April and cultivated according to common practice. Tubers were harvested in November.

### 1.1.2 Storage and Quality Measurements

Upon harvest tubers were temporarily stored at environmental temperature and after 1 week transferred to storage rooms at AFSG. Temperature during storage was set on either 4° C. or 8° C., at 95% humidity and Carvon was applied to prevent sprouting.

Sampling was performed at intake and at regular intervals during storage. The length of the intervals depended on the experiment and varied between 1 week and 3 months.

A sample was composed of at least 10 tubers randomly picked from a batch. For each sample the fry colour was determined and the concentration of sucrose, glucose and fructose, according to common procedures. Part of the sample was stored at −80° C. for future RNA analysis. This sample consisted of a combination of longitudinal ⅛th sections, including peel, form the same tubers that were used for the fry-colour and sugars determination.

1.1.3 RNA Isolation from Deep-Frozen Tuber Tissue

The deep frozen tissue from each sample was grinded and homogenized. Total RNA was isolated from the resulting powder according to Chang et al. (1993, Plant Mol. Biol. Rep. 11(2):113-116). mRNA was isolated using the Oligotex mRNA Kit (Qiagen, The Netherlands).

### 1.1.4 Micro-Array Hybridisation

Messenger RNA, up to 200 nanogram complemented with 1 nanogram luciferase polyA mRNA was used for each individual labelling. Reference RNA was labelled with Cy3 and sample RNA with Cy5 using the CyScribe First-Strand cDNA Labelling Kit (Amersham Biosciences). After labelling the sample and reference were purified with the PCR clean up kit (Qiagen, the Netherlands) After checking the integrity of the labelled cDNA using agarose electrophoresis, sample and reference cDNA were mixed and used for hybridisation of the micro-array following the protocol supplied by the manufacturer of the slides. Cover slides and hybridisation chambers from Agilent Technologies (Palo Alto) were used. Hybridisation was allowed to continue overnight in an incubator where the slides were continuously rotating (Sheldon Manufacturing). Post hybridisation washes were according to the Corning slides protocol.

### 1.1.5 Microarray Data Analysis

Slides were scanned using a GenePix 4000B (Axon Instruments) scanner and total pixel intensities were assigned to the spots using GenePixPro 6.0 software. Values were normalised by adjusting the Cy5/Cy3 ratio of medians of the luciferase signals to 1. A stringent threshold with respect to signal noise ratio and missing values was set, implying that signals not reaching 3 times local background were filtered out.

Finally duplicate expression ratios (2 log ratio Cy5/Cy3) were averaged and used for cluster analysis on the Spotfire DecisionSite for Functional Genomics (Spotfire Inc. Somerville USA, http://www.spotfire.com/products/decisionsite_microarray_analysis.cfm) and correlated with the physiological and quality data Indicator genes were selected by comparative analysis of which hexose accumulation after 3 months of storage and gene expression profiles, and confirmed and validated using the software Predicted Analysis Microarray (http://www-stat.stanford.edu/˜tibs/PAM).

### 1.1.6 Primers Development

Primers for the selected genes were designed using Primer 3 software (http://frodo.wi.mit.edu/cgi-bin/primer3/primer3_www.cgi) in combination with DNA mfo Id software (http://www.bioinfospi.edu/appliations/mfold/old/dna/).

### 1.1.7 RT-PCR

Total RNA was isolated according to the protocol described above. Preparations were DNAseI (AP Biotech) treated and purified using RNeasy (Qiagen, The Netherlands). Half a microgram of pure total RNA was used for cDNA synthesis using Anchored Oligo(dT)₂₃ (SIGMA, The Netherlands) and M-MLV Reverse Transcriptase (Invitrogen, Life Technologies). Dilutions of this cDNA were used for Realtime PCR using the qPCR Mastermix Plus for SYBR Greenl (Eurogentec, Belgium). Product formation was measured using the iCycler system (BIORAD Laboratories, The Netherlands). The signal obtained from the same batch of cDNA using primers homologous to Arabidopsis thaliana 18S rRNA was taken as a reference for normalisation. Relative changes in expression were calculated using the Gene Expression Macro (Version 1.1) supplied by BIORAD.

### 1.2 Results

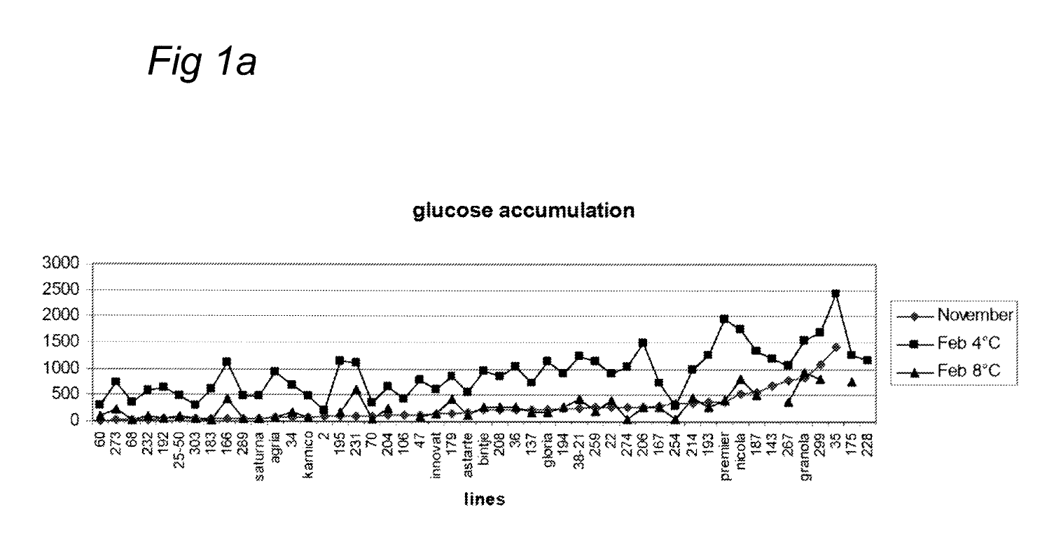

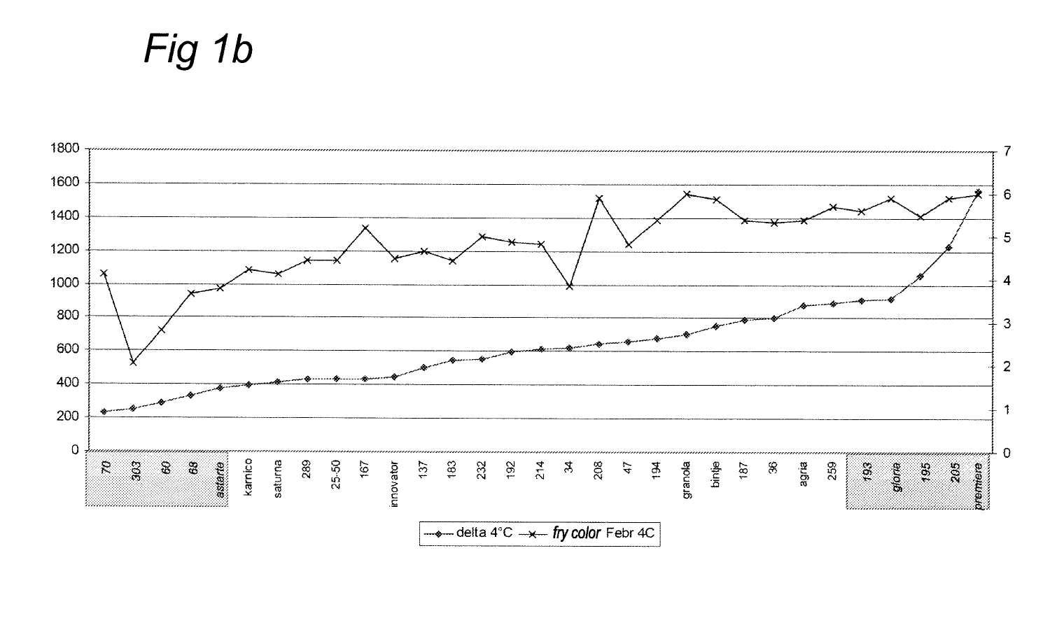

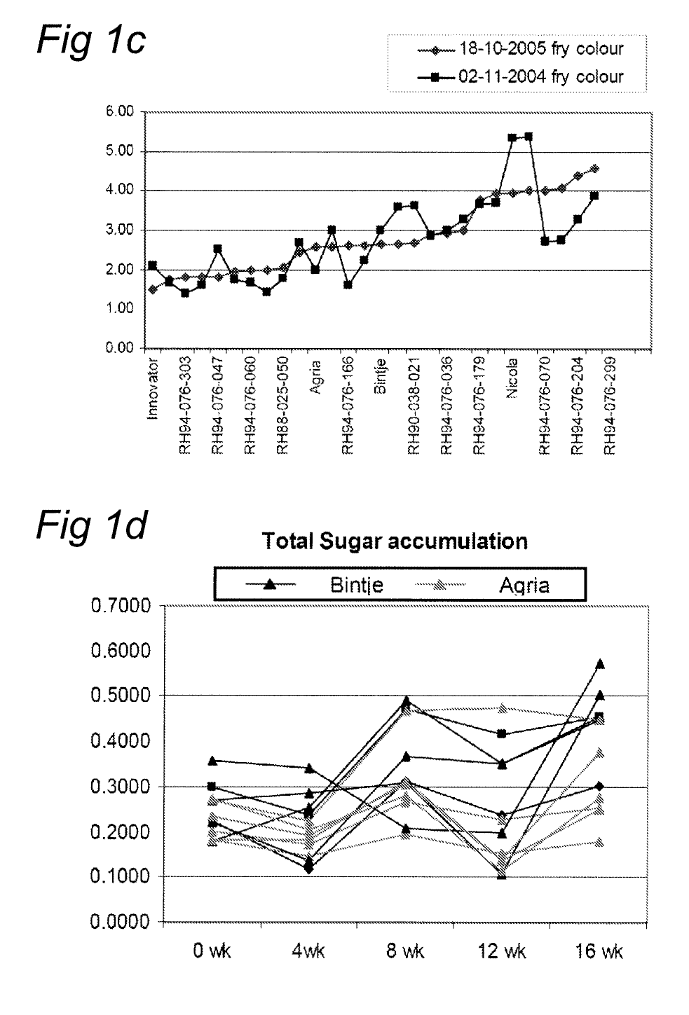

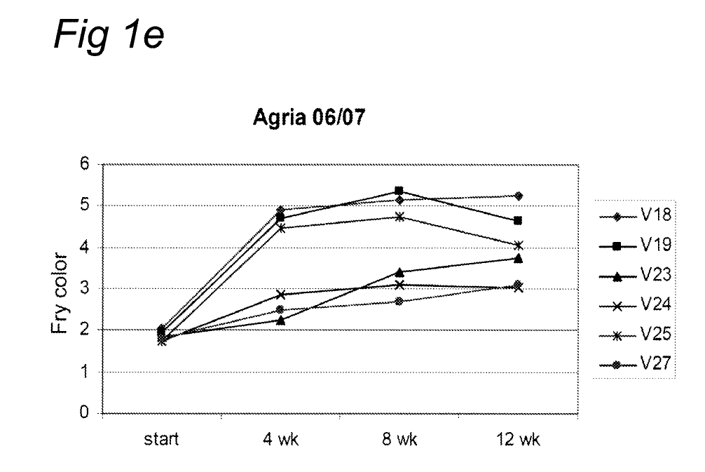

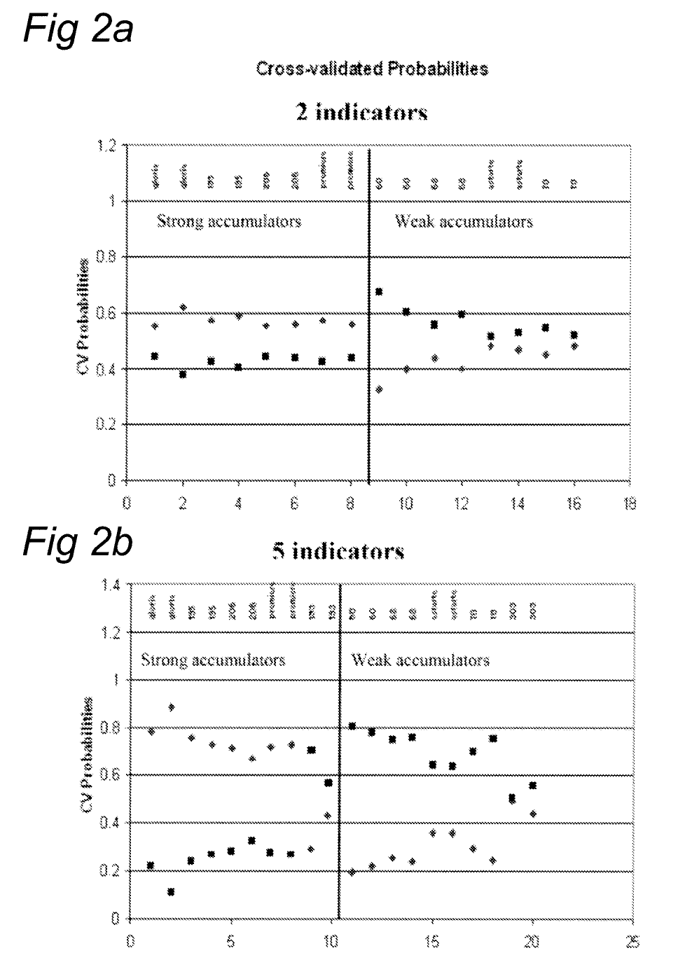

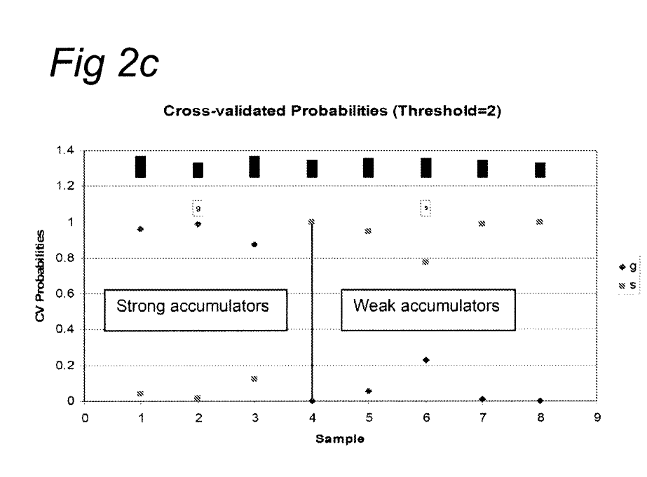

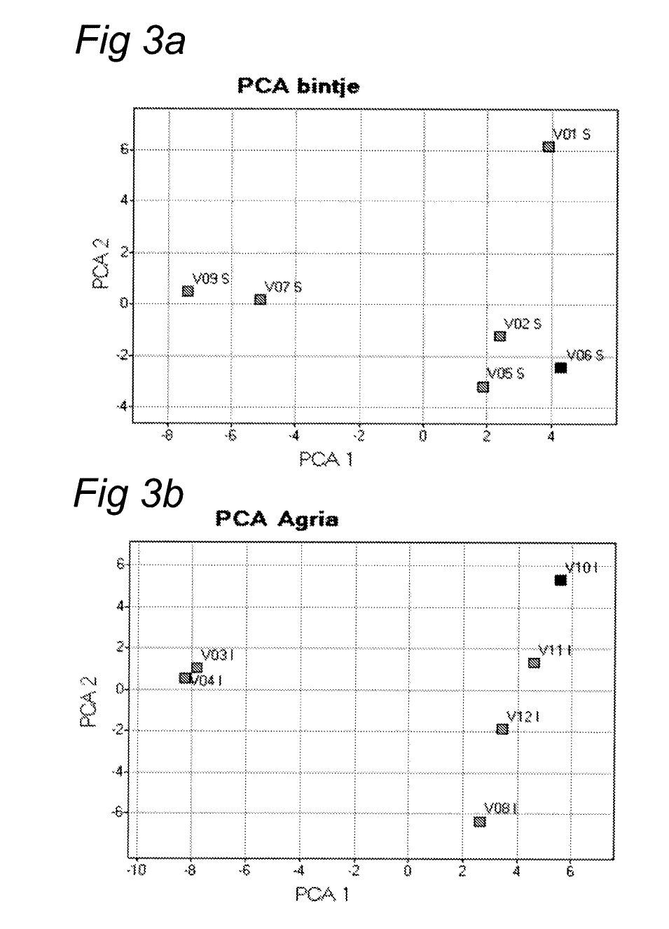

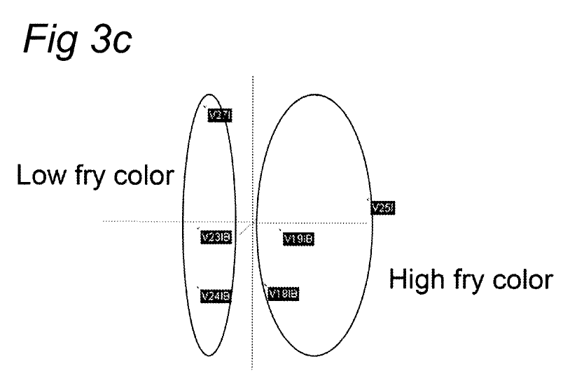

In three subsequent seasons large scale storage trials were performed including a selection of 20 or 40 segregating RH94 lines and 10 commercially used potato varieties. Sugar accumulation during storage was measured for 10 commercial potato varieties and separated in sucrose, glucose and fructose. In addition fry colour and several other quality characteristics were measured. Glucose accumulation at 4° C. after three months storage in season 2004/2005 is depicted in FIG. 1-A. For microarray analysis the most extreme lines should be identified. In order to make a relevant selection the increment in glucose concentration between 0 and 3 months (Δglucose T0/T3m) was used as a criterion. This is shown in FIG. 1-B. The 10 most extreme lines were pooled in two bulks and used for microarray analysis. Border values for glucose concentration were established for lines defined as low, medium or high sugar-accumulating. This criterion was used for all seasons and is shown in Table 1, below.

TABLE 1

| | Sugar | Storage at 4° C. | | Storage at 8° C. | |
|---|---|---|---|---|---|
| Quality | accumulation | 3 months | | 3 months | |
| class | potential | min glu* | max glu* | min glu* | max glu* |
| TOP | Low | 0.10 | 0.30 | 0.01 | 0.06 |
| MEDIUM | Medium | 0.30 | 0.50 | 0.06 | 0.10 |
| POOR | High | 0.50 | 1.00 | 0.10 | 0.20 |

*glucose concentration measured in g/100 g dry weight. At intake the concentration for all samples varied between 0.0 and 0.2 g/100 g

The quality analysis of the second storage trial revealed the same overall trend with respect to sugar accumulation as was seen in the first experiment. However, due to seasonal variation the relative quality order was not identical (FIG. 1-C).

Microarray analysis was performed in several repetitions in order to increase the validity of the resulting expression data. Repetitions included swap-dye experiments, hybridisation of the two pools against each other and hybridisation of both pools against a common reference. Expression values were validated' by analysing a small subset of genes using RT-PCR. Experiments were repeated in a second storage season with a the same lines and results were combined for cluster analysis.

This resulted in the identification of a subset of 20 genes, listed in Table 2 (SEQ ID NO: 1-20) that together best explain the difference between high accumulating and low accumulating lines over two seasons.

TABLE 2
Selection of cold sweetening indicators for quick scan at intake

| | | | Expression profile | |
|---|---|---|---|---|
| SEQ | | | low | high |
| ID | ID | Putative function | accumulators | accumulators |
| 1 | cSTB45E6 | Unknown | Low | High |
| 2 | D52 | A specific P450 monooxygenase | Low | High |
| 3 | L10B | beta-cyanoalanine synthase | Low | High |
| 4 | potato0379 | ribosomal protein ML16 | Low | High |
| 5 | potato0663 | cytochrome P450 monooxygenase | Low | High |
| 6 | potato0777 | transcription factor | Low | High |
| 7 | potato0793 | NADH2 dehydrogenase | Low | High |
| 8 | potato0921 | Tumor protein homolog (tctp) (p23) | Low | High |
| 9 | potato1234 | chloroplast small heat shock protein class I | Low | High |
| 10 | cSTA2H20 | fructose-bisphosphate aldolase | High | Low |
| 11 | cSTE7P4 | aldehyde dehydrogenase homolog | High | Low |
| 12 | cSTS19O24 | plastidic aldolase | High | Low |
| 13 | cSTS5L21 | Calmodulin | High | Low |
| 14 | cSTS6J14 | Unknown | High | Low |
| 15 | cSTS8P16 | phenylalanine ammonia-lyase | High | Low |
| 16 | potato0444 | 40S ribosomal protein S3 ( | High | Low |
| 17 | potato0782 | cytochrome P450 monooxygenase | High | Low |
| 18 | potato1439 | Ripening-related protein | High | Low |
| 19 | potato1600 | olfactory receptor | High | Low |
| 20 | potato1817 | ATP synthase beta chain | High | Low |

In addition, experiments were designed to allow prediction of intra-variety batch variation in which commercially grown Bintje and Agria, from 6 different origins and two growing seasons were analysed in the same way as described above. The results from the lab quality analysis was complemented with quality measurements (fry colour) performed in practice. Though in general, and as expected, Bintje is the stronger sugar accumulator of the two varieties, there still is a large intra-cultivar variation. As a consequence individual Bintje batches may perform better than individual Agria batches. To enhance the differences, samples from all 12 batches were cold-shocked at 2° C. for 24 hours. Gene expression profiles were taken from all batches at intake and after the cold-shock. The 24 profiles obtained each season were analysed in correlation with the results from the quality analysis and the most differential genes between good and bad performing batches were selected.

This resulted in the identification of a subset of 86 genes, listed in Table 3 that together best explain the difference between high accumulating and low accumulating batches (sequences in annex). Optimal prediction of sugar accumulation potential was obtained when the profile of the intake sample was compared to the cold-shock sample. The 86 indicator genes fall in different functional groups, and several have unknown functions.

TABLE 3
Selection of cold sweetening indicators
for use in combination with cold-shock

| SEQ ID | Id | Putative function |
|---|---|---|
| 21 | A1046 | Cis-Golgi SNARE protein |
| 22 | A461 | Patatin b2 precursor |
| 23 | A545 | Probable myosin heavy chain |
| 24 | A663 | Metallocarboxypeptidase inhibitor PFT4 |
| 25 | A665 | Cytochrome c biogenesis protein |
| 26 | A714 | Metallo carboxy peptidase inhibitor |
| 27 | A823B | Kunitz-type tuber invertase inhibitor |
| | | precursor |
| 28 | A971 | Proteinase inhibitor I |
| 29 | B10119 | Proteinase inhibitor i precursor |
| 30 | B645 | Metallocarboxypeptidase inhibitor |
| 31 | B6512B | Cytochrome P450 monooxygenase |
| 32 | B652 | Unknown |
| 33 | B809B | Unknown |
| 34 | Csta37i21 | Snakin2 |
| 35 | Cstd7g18 | Putative SCARECROW gene regulator-like |
| 36 | Cste19p23 | Cathepsin D inhibitor |
| 37 | Cste1m3 | S-adenosylmethionine decarboxylase |
| 38 | Cste21i19 | Calcium-dependent protein kinase |
| 39 | Cste21i23 | DNA binding protein EREBP-3 |
| 40 | Csts12m17 | S-adenosylmethionine synthetase 3 |
| 41 | Csts14g1 | Putative glucosyltransferase |
| 42 | Csts2n15 | SNF1-related kinase complex anchoring |
| | | protein SIP1 |
| 43 | Csts5b14 | Rrna intron-encoded homing endonuclease |
| 44 | Csts7i14 | Unknown |
| 45 | Csts7l12 | Peptide transporter |
| 46 | L10A99 | (Trans/integral)membrane protein |
| 47 | L261B | Unknown |
| 48 | L272B | Fructokinase |
| 49 | Potato0019 | Thioredoxinlike 4 |
| 50 | Potato0043 | 70 kda peptidylprolyl isomerase |
| 51 | Potato0044 | Ankyrin repeat family protein |
| 52 | Potato0086 | Ribosomal protein I28like |
| 53 | Potato0087 | Lipoxygenase |
| 54 | Potato0202 | 6 7dimethyl-ribityllumazine synthase |
| | | precursor |
| 55 | Potato0230 | Cytochrome P450 |
| 56 | Potato0239 | 60S ribosomal protein |
| 57 | Potato0249 | 40S ribosomal protein |
| 58 | Potato0320 | Adp-glucose pyrophosphorylase |
| 59 | Potato0344 | Senescence associated protein |
| 60 | Potato0370 | Unknown |
| 61 | Potato0500 | Unknown |
| 62 | Potato0612 | Unknown |
| 63 | Potato0649 | Malate dehydrogenase |
| 64 | Potato0651 | PAZ domaincontaining protein |
| 65 | Potato0667 | Cell cycle protein |
| 66 | Potato0687 | 60S ribosomal protein |
| 67 | Potato0712 | Ribosomal protein |
| 68 | Potato0760 | Ubiquitin conjugating enzyme |
| 69 | Potato0829 | Unspecific monooxygenase |
| 70 | Potato0830 | Granulebound starch synthase |
| 71 | Potato0897 | Prolinerich protein |
| 72 | Potato0906 | Aldehyde oxidase |
| 73 | Potato1043 | Cell autonomous heat shock cognate |
| | | protein 70 |
| 74 | Potato1175 | Lysosomal prox carboxypeptidase |
| 75 | Potato1209 | Oxidoreductase |
| 76 | Potato1249 | Metallocarboxypeptidase inhibitor |
| 77 | Potato1258 | Cysteine protease inhibitor 1 |
| 78 | Potato1273 | Acid invertase |
| 79 | Potato1285 | Heat shock cognate protein 80 |
| 80 | Potato1286 | Unknown |
| 81 | Potato1290 | Unknown |
| 82 | Potato1292 | Unknown |
| 83 | Potato1297 | Ubiquitin |
| 84 | Potato1304 | Retrotransposon del146 |
| 85 | Potato1317 | Casein kinase |
| 86 | Potato1333 | Unknown |
| 87 | Potato1357 | Cytochrome P450 monooxygenase |
| 88 | Potato1464 | Unknown |
| 89 | Potato1662 | Unknown |
| 90 | Potato1669 | Phosphatidyl choline 2 acylhydrolase |
| 91 | Potato1747 | Cyc07 |
| 92 | Potato1749 | Fructose1 6 bisphosphate aldolase |
| 93 | Potato1761 | Dehydration responsive protein |
| 94 | Potato1796 | Rna binding protein |
| 95 | B644 | Metallo carboxy peptidase inhibitor |
| 96 | BK F4 | metallo carboxy peptidase inhibitor |
| 97 | BK T2 | metallo carboxy peptidase |
| 98 | potato0833 | putative senescence associated protein |
| | | [Pisum sativum] |
| 99 | potato1775 | hypothetical protein ARG10 mung bean |
| | | ARG10 [Vigna radiata] |
| 100 | potato1165 | unknown protein [Arabidopsis thaliana] |
| 101 | potato0340 | 5lipoxygenase [Solanum tuberosum] |
| 102 | A832 | PROTEINASE INHIBITOR I PRECURSOR |
| 103 | potato0732 | unknown protein [Arabidopsis thaliana] |
| 104 | potato1546 | cellulose synthase [Solanum tuberosum] |
| 105 | potato1650 | unknown |
| 106 | potato1587 | histone 3 |

Subsets of the 106 genes represented in Tables 2 and 3 were translated into RT-PCR assays and these RT-PCR primers were used to validate the microarray results on individual pool-members, for instance on SEQ ID NO: 78, 86, 89, 95, 96, 97, 98, 99, 100, 101, 102, 103, 104, 105 and 106 and independent samples of for instance Agria batches of two different storage seasons. As expected the correlation was lost when individual batches were screened with single genes. However, careful selection allowed the identification of subsets of multiple genes that regained the correlation demonstrated in the microarray analysis of pooled batches. The predictive value increases when larger subsets are used. It was demonstrated using PAM analysis that these subsets can be used to predict cold sweetening during storage in individual batches. An example PAM analysis is shown in FIG. 2.

As for the multi-batch trials using Bintje and Agria, PCA plots also show that the selected genes can be used to separate the high-performing batches from the low performers, both for Agria and Bintje (FIG. 3). The plots demonstrate that the selected indicator genes are able to discriminate between batches before storage. The groups are in agreement with the quality classes designated after storage on the basis of sugars content and frying colour:

| Q-class | Bintje | Agria |
|---|---|---|
| 1 | V01 | V03 |
| | | V04 |
| | | V27I |
| | | V23IB |
| | | V24IB |
| 2 | V09 | V12 |
| | V07 | V11 |
| | | V10 |
| 3 | V06 | V08 |
| | V05 | V251 |
| | V02 | V19IB |
| | | V18IB |

### CONCLUSIONS

The selected set of 106 indicator genes and subsets thereof can be used as a diagnostic tool to predict cold sweetening potential of potatoes for at least 3 months and longer, since in general sugar-starch metabolism is most active in the first 3 months of storage. Quick prediction at intake can be performed on single samples using subsets from genes summarized in Table 2, and/or variants thereof. However, a more robust distinction between high and low quality batches is obtained when a 1-day cold shock is applied and a comparison is made between the initial sample and the cold shocked sample. In that case a selection of the genes from Table 3 may be used (and/or variants thereof).

The indicators can be used to assist storage planning according to the FEFO principle (first expired, first out) since it allows identification of high risk batches that should be processed first. In addition the test can be used for processing planning for the frying industry. Batches with comparable sugar accumulating potential can be sorted out and processed in one string. This will reduce the number of shifts in processing parameters and will therefore enhance the cost-efficiency.

1. A method for determining a cold-storage induced sweetening stage and/or a sweetening potential of one or more potato tuber batches comprising the steps:
(a) providing or receiving a nucleic acid sample from a batch of potato tubers,
(b) analyzing the nucleic acid sample by determining an expression profile of a set of indicator mRNA transcripts in the sample, which is indicative of a sweetening potential of the batch, and optionally
(c) designating the batch for further use.

2. The method according to claim 1, wherein the nucleic acid sample is obtained from at least 5 different potato tubers of said batch.

3. The method according to claim 1, wherein the batch is designated for further processing if the expression profile correlates with a high sweetening potential or wherein the batch is designated for cold storage if the expression profile correlates with a low sweetening potential.

4. The method according to claim 1, whereby the expression profile of at least 2, preferably at least 3, different indicator mRNA transcripts in said nucleic acid sample is determined.

5. The method according to claim 4, whereby the expression profile of at least 5, preferably at least 10 different indicator mRNA transcripts in said nucleic acid sample is determined.

6. The method according to claim 5, wherein said indicator mRNA transcripts are selected from the group of nucleic acid sequences consisting of SEQ ID NO: 1 to SEQ ID NO: 106, their complements, or their nucleic acid homologs comprising at least 70% sequence homology over the entire length of said sequences.

7. The method according to claim 6, wherein fragments of said nucleic acid sequences, their complements, or their nucleic acid homologs are used for the determination of a cold-storage induced sweetening stage and/or the sweetening potential of one or more batches of potato tubers or portions thereof.

8. The method according to claim 7, wherein said fragments are at least 10 nucleotides in length.

9. A solid carrier comprising at least 3 nucleic acid molecules attached to said carrier, said at least 3 nucleic acid molecules being selected from the group of nucleic acid sequences consisting of SEQ ID NO: 1 to SEQ ID NO: 106, their complements, or their nucleic acid homologs comprising at least 70% sequence homology over the entire length of said sequences.

10. The solid carrier according to claim 9, wherein said at least 3 nucleic acid homologs are capable of hybridizing under stringent conditions to at least 3 nucleic acid molecules selected from the group of nucleic acid sequences consisting of SEQ ID NO: 1 to SEQ ID NO: 106 or their complements.

11. The carrier according to claim 9, wherein the carrier comprises one or more of the following: glass, plastic, nitrocellulose, nylon or silicon.

12. A kit for determining a cold-storage induced sweetening stage and/or sweetening potential of a potato tissue sample, said kit comprising nucleic acid probes or primers capable of detecting the presence and/or quantity of at least 3 nucleic acid molecules within a set of nucleic acid molecules, said set being selected from the group of nucleic acid sequences consisting of SEQ ID NO: 1 to SEQ ID NO: 106, their complements, or their nucleic acid homologs comprising at least 70% sequence homology over the entire length of said sequences.

13. The kit according to claim 12, further comprising one or more of the following: instructions for use, control samples, control data, labeling reagents, detection reagents, hybridization or amplification reagents, primers or probes for detecting housekeeping-gene transcripts, containers or carriers.
